(19) United States
(12) Patent Application Publication
(10) Pub. No.: US 2025/0340527 A1
(43) Pub. Date: Nov. 6, 2025

(54) RIPK2 AND NOD INHIBITORS

(71) Applicants: Trustees of Tufts College, Medford, MA (US); University of Houston System, Houston, TX (US)

(72) Inventors: Gregory Cuny, Houston, TX (US); Shirin Shabahang, Houston, TX (US); Alexei Degterev, Brookline, MA (US)

(21) Appl. No.: 18/866,426

(22) PCT Filed: May 18, 2023
(86) PCT No.: PCT/US2023/067181

§ 371 Date: Nov. 15, 2024

Related U.S. Application Data
(60) Provisional application No. 63/343,343, filed on May 18, 2022.

(51) Int. Cl. C07D 401/10; A61K 31/4418; A61K 31/4545; A61K 31/496; A61P 25/28; C07D 213/61

(52) U.S. Cl. CPC C07D 401/10; A61K 31/4418; A61K 31/4545; A61K 31/496; A61P 25/28; C07D 213/61

(57) ABSTRACT

Substituted phenylpyridine compounds are disclosed. Also disclosed is the use of the phenylpyridine compounds in methods of treatment and for inhibiting RIPK2 and/or NOD2 cell signaling.

### CROSS-REFERENCE TO RELATED APPLICATIONS

The present application claims priority to U.S. Provisional Patent Application No. 63/343,343, filed May 18, 2022, the entire contents of which are hereby incorporated by reference.

### STATEMENT REGARDING FEDERALLY SPONSORED RESEARCH

This invention was made with government support under NS111395 awarded by the National Institutes of Health. The government has certain rights in the invention.

### BACKGROUND OF THE INVENTION

Protein kinases are important enzymes in cellular signal transduction. In many pathological conditions aberrant signal transduction occurs. Therefore, protein kinases can be used as therapeutic agents for the treatment of various diseases. Receptor interacting kinase 2 (RIPK2) and/or nucleotide-binding oligomerization domain (NOD) cell signaling may mediate pro-inflammatory signaling and may be therapeutic targets in autoimmune and inflammatory diseases, such as inflammatory bowel disease (IBD), neuropathic pain, arthritis, psoriasis, and multiple sclerosis. RIPK2 and NOD may also be targets for treating cancer. As a result, there is a need for RIPK2 or NOD inhibitors.

### BRIEF SUMMARY OF THE INVENTION

Substituted phenylpyridine compounds having a formula of

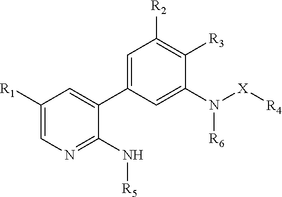

and pharmaceutically acceptable salts thereof are disclosed. R₁ may be selected from

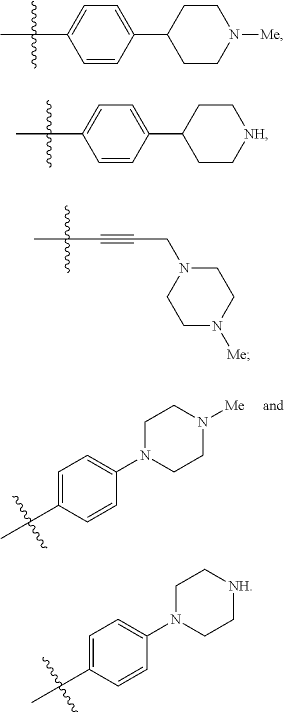

R₂ may be selected from H, halo, C₁-C₃ alkoxyl optionally substituted with halo, and —O-phenyl. R₃ may be selected from H, halo, C₁-C₃ alkoxyl optionally substituted with halo, and C₁-C₃ alkyl. R₄ may be selected from C₁-C₃ alkyl optionally substituted with halo, phenyl, and benzyl. R₅ may be selected from H and methyl. R₆ may be selected from H and methyl. X may be selected from SO₂ and CO. Each of R₁, R₂, R₃, R₄, R₅, R₆, and X may be independently selected. The compounds disclosed herein may be capable of inhibiting a protein kinase and/or NOD cell signaling. Particularly, the compounds disclosed herein may be capable of inhibiting RIPK2 and/or NOD2 cell signaling.

Pharmaceutical compositions comprising the compounds or pharmaceutically acceptable salts thereof are also provided.

Another aspect of the technology provides for methods of treatment. Treatment may be affected by administering a compound described herein to a subject in need of treatment. The subject in need treatment may be one in need of inhibition of a protein kinase and/or NOD cell signaling. The subject in need treatment may be one in need of inhibition of a RIPK2 and/or NOD2 cell signaling. In some instances, the subject is in need of a treatment for an autoimmune or inflammatory disease, such as multiple sclerosis.

Another aspect of the technologies provides for methods of inhibiting a protein kinase. The method may comprise contacting the protein kinase, such as RIPK2, with a compound or pharmaceutically acceptable salt thereof as described herein. Contact between the protein kinase and the compound or salt may occur in vivo, in vitro, or ex vivo. Where contact is in vivo, a subject may be administered an effective amount of the compound or pharmaceutically acceptable salt to treat a disease or disorder associated with protein kinase activity.

Another aspect of the technologies provides for methods of inhibiting NOD cell signaling. The method may comprise contacting a cell expressing a NOD protein with a compound or pharmaceutically acceptable salt thereof as described herein. Contact between the cell and the compound or salt may occur in vivo, in vitro, or ex vivo. Where contact is in vivo, a subject may be administered an effective amount of the compound or pharmaceutically acceptable salt to treat a disease or disorder associated with NOD cell signaling activity.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1 illustrates the comparison between SS-1-64 and vehicle control in experimental autoimmune encephalomyelitis (EAE) model of multiple sclerosis.

### DETAILED DESCRIPTION OF THE INVENTION

Disclosed herein are compounds capable of inhibiting a protein kinase, such as RIPK2, or NOD cell signaling. The compounds may be used to inhibit RIPK2 and/or NOD2 cell signaling and be effective in treating diseases or disorders associated therewith.

The NOD proteins 1 and 2 are members of the NOD-like receptor (NLR) family that are involved in the innate immune system's detection of bacterial peptidoglycan (PG) derivatives. NOD1 is stimulated by bacterial PG fragments containing diaminopimelic acid (DAP), while NOD2 senses muramyl dipeptide (MDP). NOD1/2 then initiates assembly of signaling complexes by oligomerization through the nucleotide-binding oligomerization domains (NBD), which triggers the recruitment of interacting proteins through homotypic caspase-activated recruitment domain (CARD)-mediated interactions. RIPK2 is one of the key molecules in NOD-dependent signaling as it plays a role in the activation of NF-κB pathway and mitogen-activated protein kinase (MAPK) pathways that ultimately lead to synthesis of pro-inflammatory cytokines and antimicrobial molecules. Aberrant RIPK2-NOD signaling pathways plays a role in various autoimmune or inflammatory diseases or disorders. More particularly, positive or negative dysregulation of the NOD2-dependent signaling pathway has been shown to facilitate several chronic inflammatory disorders.

As demonstrated in the Examples, the presently disclosed compounds inhibit RIPK2 and/or NOD2 cell signaling, have good pharmacokinetics, and may be used to reduce paralytic symptoms in an experimental autoimmune encephalomyelitis (EAE) model of multiple sclerosis.

### Compounds

The compounds disclosed herein are substituted phenylpyridine compounds having a formula of

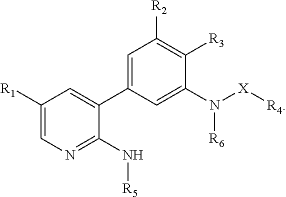

R₁ may be selected from

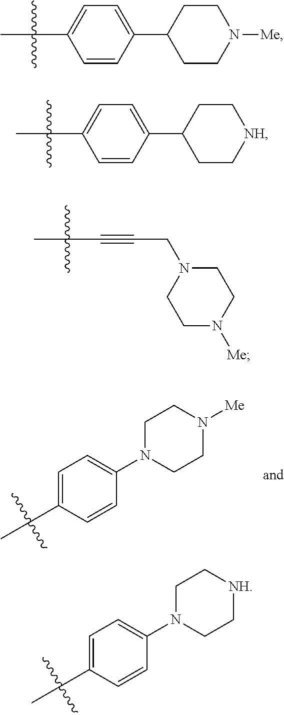

In some embodiments, R₁ is

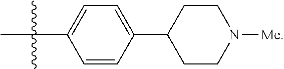 [SMILES: *c1ccc(C2CCN(C)CC2)cc1]

In some embodiments, R₁ is

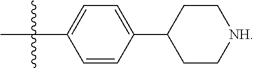 [SMILES: *c1ccc(C2CCNCC2)cc1]

In some embodiments, R₁ is

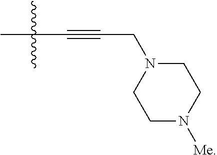 [SMILES: *C#CCN1CCN(C)CC1]

In some embodiments, R₁ is

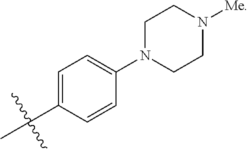 [SMILES: *c1ccc(N2CCN(C)CC2)cc1]

In some embodiments, R₁ is

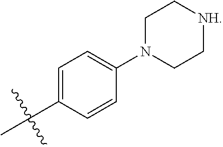 [SMILES: *c1ccc(N2CCNCC2)cc1]

R₂ may be selected from H, halo, C₁-C₃ alkoxyl optionally substituted with halo, and —O-phenyl. Exemplary R₂ include, without limitation, hydrogen, fluoro (F), chloro (Cl), methoxy (OMe), ethoxy (OEt), n-propoxy (O-nPr), i-propoxy (O-iPr), trifluoromethoxy (OCF₃), or O-phenyl (OPh). In some embodiments, R₂ is methoxy. In other embodiments, R₂ is fluoro.

R₃ may be selected from H, halo, C₁-C₃ alkoxyl optionally substituted with halo, and C₁-C₃ alkyl. Exemplary R₃ include, without limitation, hydrogen, fluoro (F), chloro (Cl), methoxy (OMe), ethoxy (OEt), n-propoxy (O-nPr), i-propoxy (O-iPr), trifluoromethoxy (OCF₃), methyl (Me), ethyl (Et), n-propyl (n-Pr), and i-propyl (i-Pr). In some embodiments, R₃ is methoxy.

R₄ may be selected from C₁-C₃ alkyl optionally substituted with halo, phenyl, and benzyl. Exemplary R₄ include, without limitation, methyl (Me), ethyl (Et), n-propyl (n-Pr), i-propyl (i-Pr), trifluoromethyl (CF₃), difluoropropyl (e.g., CF₂Et), phenyl (Ph), and benzyl (CH₂Ph). In some embodiments, R₄ is n-Pr. In some embodiments, R₄ is methyl.

R₅ may be selected from H and methyl. In some embodiments, R₅ is H. In some embodiments, R₅ is methyl.

R₆ may be selected from H and methyl. In some embodiments, R₆ is H. In some embodiments, R₆ is methyl.

X may be selected from SO₂ and CO. In some embodiments, X is SO₂. In some embodiments, X is CO.

In some embodiments, R₅ or R₆ is methyl. In other embodiments, if each of R₅ and R₆ are hydrogen, then R₄ is methyl or X is CO.

In some embodiments where R₁ is

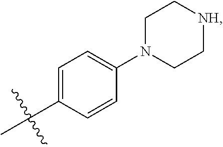 [SMILES: *c1ccc(N2CCNCC2)cc1]

R₅ is methyl, R₆ is methyl, R₄ is methyl, or X is CO. In some embodiments, at least one of R₅ or R₆ is methyl. In some embodiments, each of R₅ and R₆ are hydrogen, then R₄ is methyl or X is CO.

Exemplary compounds include, without limitation, one or more of.
N-(3-fluoro-2-methoxy-5-(2-(methylamino)-5-(4-(piperazin-1-yl)phenyl)pyridin-3-yl)phenyl)propane-1-sulfonamide (SS-1-10),
N-(5-(2-amino-5-(4-(piperidin-4-yl)phenyl)pyridin-3-yl)-3-fluoro-2-methoxyphenyl)propane-1-sulfonamide (SS-1-14),
N-(5-(2-amino-5-(4-(4-methylpiperazin-1-yl)phenyl)pyridin-3-yl)-3-fluoro-2-methoxyphenyl)propane-1-sulfonamide (SS-1-18),
N-(5-(2-amino-5-(4-(piperazin-1-yl)phenyl)pyridin-3-yl)-3-fluoro-2-methoxyphenyl)methanesulfonamide (SS-1-33),
N-(5-(2-amino-5-(4-(1-methylpiperidin-4-yl)phenyl)pyridin-3-yl)-3-fluoro-2-methoxyphenyl)propane-1-sulfonamide (SS-1-38),
N-(5-(2-amino-5-(4-(piperazin-1-yl)phenyl)pyridin-3-yl)-3-fluoro-2-methoxyphenyl)-N-methylpropane-1-sulfonamide (SS-1-39),
N-(5-(2-amino-5-(4-(1-methylpiperidin-4-yl)phenyl)pyridin-3-yl)-3-fluoro-2-methoxyphenyl)methanesulfonamide (SS-1-43),
N-(5-(2-amino-5-(4-(1-methylpiperidin-4-yl)phenyl)pyridin-3-yl)-3-fluoro-2-methoxyphenyl)-N-methylmethanesulfonamide (SS-1-50),
N-(5-(2-amino-5-(4-(1-methylpiperidin-4-yl)phenyl)pyridin-3-yl)-2,3-dimethoxyphenyl)methanesulfonamide (SS-1-64),
N-(5-(2-amino-5-(3-(4-methylpiperazin-1-yl)prop-1-yn-1-yl)pyridin-3-yl)-3-fluoro-2-methoxyphenyl)propane-1-sulfonamide (SS-1-66), and
N-(5-(2-amino-5-(4-(1-methylpiperidin-4-yl)phenyl)pyridin-3-yl)-2,3-dimethoxyphenyl)acetamide (SS-2-41).

Exemplary methods for preparing the compounds disclosed herein are provided in the Examples. Compounds having a R₁ group comprising phenyl may be prepared by reacting a substituted phenylboronic acid pinacol ester with a substituted bromopyridine. Compounds having a R₁ group comprising an alkyne may be prepared by reacting a substituted alkyne with a substituted bromopyridine.

The term “optionally substituted” refers to one or more carbon atoms in the group being independently substituted with one or more functional groups described herein.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” or “+” or “−” depending on the configuration of substituents around the stereogenic carbon atom and or the optical rotation observed. The present invention encompasses various stereo isomers of these compounds and mixtures thereof. Stereoisomers include enantiomers and diastereomers. Mixtures of enantiomers or diastereomers may be designated (±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise. Also contemplated herein are compositions comprising, consisting essentially of, or consisting of an enantiopure compound, which composition may comprise, consist essentially of, or consist of at least about 50%, 60%, 70%, 80%, 90%, 95%, 96%, 97%, 98%, 99%, or 100% of a single enantiomer of a given compound (e.g., at least about 99% of an R enantiomer of a given compound).

As used herein, “salt” refers to acid addition salts and basic addition salts. It may also refer to those salts that may be prepared in situ during the final isolation and purification of the compounds of the invention.

Examples of acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isothionate), lactate, malate, maleate, methanesulfonate, nicotinate, 2-naphthalenesulfonate, oxalate, palmitate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups may be quaternized with such agents as lower alkyl halides such as, but not limited to, methyl, ethyl, propyl, and butyl chlorides, bromides and iodides; dialkyl sulfates like dimethyl, diethyl, dibutyl and diamyl sulfates; long chain halides such as, but not limited to, decyl, lauryl, myristyl and stearyl chlorides, bromides and iodides; arylalkyl halides like benzyl and phenethyl bromides and others. Water or oil-soluble or dispersible products are thereby obtained. Examples of acids which may be employed to form pharmaceutically acceptable acid addition salts include such inorganic acids as hydrochloric acid, hydrobromic acid, sulfuric acid, and phosphoric acid and such organic acids as acetic acid, fumaric acid, maleic acid, 4-methylbenzenesulfonic acid, succinic acid, and citric acid.

Basic addition salts may be prepared in situ during the final isolation and purification of compounds of this invention by reacting a carboxylic acid-containing moiety with a suitable base such as, but not limited to, the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as, but not limited to, lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other examples of organic amines useful for the formation of base addition salts include ethylenediamine, ethanolamine, diethanolamine, piperidine, piperazine and the like.

Compounds described herein may exist in unsolvated as well as solvated forms, including hydrated forms, such as hemi-hydrates. In general, the solvated forms, with pharmaceutically acceptable solvents such as water and ethanol among others are equivalent to the unsolvated forms for the purposes of the invention.

The disclosed compounds may exhibit one or more biological activities. In some embodiments, the disclosed compounds modulate the activity of a protein kinase, such as RIPK2. In some embodiments, the disclosed compounds inhibit the activity of RIPK2 by at least 50%, 60%, 70%, 80%, 90%, 95%, 96%, 97%, 98%, or 99% at a concentration of less than 100 μM, 50 μM, 10 μM, 1 μM, 0.1 μM, 0.05 μM, 0.01 μM, 0.005 μM, 0.001 μM, or 0.0001 μM less. In some embodiments, the compounds disclosed herein have an IC₅₀ less than 200 nM, 150 nM, 100 nM, 75 nM, 50 nM, 40 nM, 30 nM, or 20 nM as measured by a RIPK2 enzyme assay such as described in Example 2.

The disclosed compounds may be used to inhibit a protein kinase, such as RIPK2, by contacting a compound, or salt thereof, as disclosed herein with the protein kinase. The contacting step may be performed in vitro, ex vivo, or in vivo.

In some embodiments, the disclosed compounds modulate the activity of NOD, e.g., NOD2, cell signaling. In some embodiments, the disclosed compounds inhibit the activity of NOD cell signaling by at least 50%, 60%, 70%, 80%, 90%, 95%, 96%, 97%, 98%, or 99% at a concentration of less than 100 μM, 50 μM, 10 μM, 1 μM, 0.1 μM, 0.05 μM, 0.01 μM, 0.005 μM, 0.001 μM, or 0.0001 μM less. In some embodiments, the compounds disclosed herein have an IC₅₀ less than 75 nM, 50 nM, 25 nM, 10 nM, 5 nM, 2 nM, 1 nM, 0.5 nM, 0.2 nM, 0.1 nM, 0.05 nM, or 0.02 nM as measured by a NOD2 signaling assay such as described in Examples 3 or 4.

The disclosed compounds may be used to inhibit NOD cell signaling by contacting a compound, or salt thereof, as disclosed herein with a cell expressing a NOD protein, e.g., NOD2. The contacting step may be performed in vitro, ex vivo, or in vivo.

### Pharmaceutical Compositions

The compounds employed in the compositions and methods disclosed herein may be administered as pharmaceutical compositions and, therefore, pharmaceutical compositions incorporating the compounds are considered to be embodiments of the compositions disclosed herein. Such compositions may take any physical form which is pharmaceutically acceptable; illustratively, they can be orally administered pharmaceutical compositions. Such pharmaceutical compositions contain an effective amount of a disclosed compound, which effective amount is related to the daily dose of the compound to be administered. Each dosage unit may contain the daily dose of a given compound or each dosage unit may contain a fraction of the daily dose, such as one-half or one-third of the dose. The amount of each compound to be contained in each dosage unit can depend, in part, on the identity of the particular compound chosen for the therapy and other factors, such as the indication for which it is given. The pharmaceutical compositions disclosed herein may be formulated so as to provide quick, sustained, or delayed release of the active ingredient after administration to the patient by employing well known procedures.

In some embodiments, the compounds disclosed herein may be formulated as pharmaceutical compositions that include: (a) a therapeutically effective amount of one or more compounds as disclosed herein; and (b) one or more pharmaceutically acceptable carriers, excipients, or diluents. The pharmaceutical composition may include the compound in a range of about 0.1 to 2000 mg (preferably about 0.5 to 500 mg, and more preferably about 1 to 100 mg). The pharmaceutical composition may be administered to provide the compound at a daily dose of about 0.1 to about 1000 mg/kg body weight (preferably about 0.5 to about 500 mg/kg body weight, more preferably about 50 to about 100 mg/kg body weight). In some embodiments, after the pharmaceutical composition is administered to a subject (e.g., after about 1, 2, 3, 4, 5, or 6 hours post-administration), the concentration of the compound at the site of action may be within a concentration range bounded by end-points selected from 0.00 μM, 0.00 μM, 0.01 μM, 0.5 μM, 0.1 μM, 1.0 μM, 10 μM, and 100 μM (e.g., 0.1 μM-1.0 μM).

It is understood by those skilled in the art that dosage amount will vary with the activity of a particular inhibitor compound, disease state, route of administration, duration of treatment, and like factors well-known in the medical and pharmaceutical arts. In general, a suitable dose will be an amount which is the lowest dose effective to produce a therapeutic or prophylactic effect. If desired, an effective dose of such a compound, pharmaceutically acceptable salt thereof, or related composition may be administered in two or more sub-doses, administered separately over an appropriate period of time.

The compounds utilized in the methods disclosed herein may be formulated as a pharmaceutical composition that includes a carrier. For example, the carrier may be selected from the group consisting of proteins, carbohydrates, sugar, talc, magnesium stearate, cellulose, calcium carbonate, and starch-gelatin paste.

Suitable diluents may include pharmaceutically acceptable inert fillers, such as microcrystalline cellulose, lactose, dibasic calcium phosphate, saccharides, and mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

The compounds utilized in the methods disclosed herein may be formulated as a pharmaceutical composition that includes one or more binding agents, filling agents, lubricating agents, suspending agents, sweeteners, flavoring agents, preservatives, buffers, wetting agents, disintegrants, and effervescent agents.

Filling agents may include lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™).

Suitable lubricants, including agents that act on the flowability of the powder to be compressed, may include colloidal silicon dioxide, such as Aerosil® 200, talc, stearic acid, magnesium stearate, calcium stearate, and silica gel.

Examples of sweeteners may include any natural or artificial sweetener, such as sucrose, xylitol, sodium saccharin, cyclamate, aspartame, and acsulfame.

Examples of flavoring agents are Magnasweet® (trademark of MAFCO), bubble gum flavor, and fruit flavors, and the like. Examples of preservatives may include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

The compounds utilized in the methods disclosed herein may be administered in conventional dosage forms prepared by combining the active ingredient with standard pharmaceutical carriers or diluents according to conventional procedures well known in the art. These procedures may involve mixing, granulating and compressing or dissolving the ingredients as appropriate to the desired preparation.

Pharmaceutical compositions comprising the compounds may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis.

Pharmaceutical compositions adapted for topical administration may be formulated as ointments, creams, suspensions, lotions, powders, solutions, pastes, gels, impregnated dressings, sprays, aerosols or oils and may contain appropriate conventional additives such as preservatives, solvents to assist drug penetration and emollients in ointments and creams.

For applications to the eye or other external tissues, for example the mouth and skin, the pharmaceutical compositions are in some embodiments applied as a topical ointment or cream. When formulated in an ointment, the compound may be employed with either a paraffinic or a water-miscible ointment base. Alternatively, the compound may be formulated in a cream with an oil-in-water cream base or a water-in-oil base. Pharmaceutical compositions adapted for topical administration to the eye include eye drops where the active ingredient is dissolved or suspended in a suitable carrier, especially an aqueous solvent.

Pharmaceutical compositions adapted for topical administration in the mouth include lozenges, pastilles and mouth washes.

Pharmaceutical compositions adapted for rectal administration may be presented as suppositories or enemas.

Pharmaceutical compositions adapted for nasal administration where the carrier is a solid include a coarse powder having a particle size (e.g., in the range 20 to 500 microns) which is administered in the manner in which snuff is taken (i.e., by rapid inhalation through the nasal passage from a container of the powder held close up to the nose). Suitable formulations where the carrier is a liquid, for administration as a nasal spray or as nasal drops, include aqueous or oil solutions of the active ingredient.

Pharmaceutical compositions adapted for administration by inhalation include fine particle dusts or mists which may be generated by means of various types of metered dose pressurized aerosols, nebulizers or insufflators.

Pharmaceutical compositions adapted for vaginal administration may be presented as pessaries, tampons, creams, gels, pastes, foams or spray formulations.

Pharmaceutical compositions adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tableting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminum stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavoring or coloring agents.

Optionally, the disclosed compounds or pharmaceutical compositions comprising the disclosed compounds may be administered with additional therapeutic agents. In some embodiments of the disclosed methods, one or more additional therapeutic agents are administered with the disclosed compounds or with pharmaceutical compositions comprising the disclosed compounds, where the additional therapeutic agent is administered prior to, concurrently with, or after administering the disclosed compounds or the pharmaceutical compositions comprising the disclosed compounds. In some embodiments, the disclosed pharmaceutical composition is formulated to comprise the disclosed compounds and further to comprise one or more additional therapeutic agents.

Methods of preparing pharmaceutical formulations or compositions include the step of bringing an inhibitor compound into association with a carrier and, optionally, one or more additional adjuvants or ingredients. For example, standard pharmaceutical formulation techniques can be employed, such as those described in Remington's Pharmaceutical Sciences, Mack Publishing Company, Easton, PA.

Regardless of composition or formulation, those skilled in the art will recognize various avenues for medicament administration, together with corresponding factors and parameters to be considered in rendering such a medicament suitable for administration.

### Methods

The disclosed compounds and pharmaceutical compositions comprising the disclosed compounds may be administered in methods of treating a subject in need thereof. For example, in the methods of treatment a subject in need thereof may include a subject having an autoimmune and inflammatory diseases, such as inflammatory bowel disease (IBD), neuropathic pain, arthritis, psoriasis, or multiple sclerosis, or a cell proliferative disease, disorder, or condition such as cancer.

As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration.

A “subject in need thereof” as utilized herein refers to a subject in need of treatment for a disease or disorder associated with protein kinase, e.g., RIPK2, and/or NOD2 cell signaling activity. The term “subject” may be used interchangeably with the terms “individual” and “patient” and includes human and non-human mammalian subjects. In some embodiments, the treated subject may be a mammalian subject. Although the methods disclosed herein are particularly intended for the treatment of humans, other mammals are included. By way of non-limiting examples, mammalian subjects include monkeys, equines, cattle, canines, felines, mice, rats and pigs.

As used herein, the term “disorder” refers to a condition in which there is a disturbance of normal functioning. A “disease” is any abnormal condition of the body or mind that causes discomfort, dysfunction, or distress to the person affected or those in contact with the person. Sometimes the term is used broadly to include injuries, disabilities, syndromes, symptoms, deviant behaviors, and atypical variations of structure and function, while in other contexts these may be considered distinguishable categories. It should be noted that the terms “disease”, “disorder”, “condition” and “illness”, are equally used herein.

The compounds for use according to the methods of disclosed herein may be administered as a single compound or a combination of compounds. For example, a compound that modulates protein kinase, e.g., RIPK2, and/or NOD2 activity may be administered as a single compound or in combination with another compound that modulates protein kinase, e.g., RIPK2, and/or NOD2 activity or that has a different pharmacological activity.

In some embodiments of the disclosed treatment methods, the subject may be administered a dose of a compound as low as 1.25 mg, 2.5 mg, 5 mg, 7.5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, 50 mg, 52.5 mg, 55 mg, 57.5 mg, 60 mg, 62.5 mg, 65 mg, 67.5 mg, 70 mg, 72.5 mg, 75 mg, 77.5 mg, 80 mg, 82.5 mg, 85 mg, 87.5 mg, 90 mg, 100 mg, 200 mg, 500 mg, 1000 mg, or 2000 mg once daily, twice daily, three times daily, four times daily, once weekly, twice weekly, or three times per week in order to treat the disease or disorder in the subject. In some embodiments, the subject may be administered a dose of a compound as high as 1.25 mg, 2.5 mg, 5 mg, 7.5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, 50 mg, 52.5 mg, 55 mg, 57.5 mg, 60 mg, 62.5 mg, 65 mg, 67.5 mg, 70 mg, 72.5 mg, 75 mg, 77.5 mg, 80 mg, 82.5 mg, 85 mg, 87.5 mg, 90 mg, 100 mg, 200 mg, 500 mg, 1000 mg, or 2000 mg, once daily, twice daily, three times daily, four times daily, once weekly, twice weekly, or three times per week in order to treat the disease or disorder in the subject. Minimal and/or maximal doses of the compounds may include doses falling within dose ranges having as end-points any of these disclosed doses (e.g., 2.5 mg-200 mg).

In some embodiments of the disclosed treatment methods, a minimal dose level of a compound for achieving therapy in the disclosed methods of treatment may be at least about 10, 20, 30, 40, 50, 60, 70, 80, 90, 100, 150, 200, 250, 300, 350, 400, 450, 500, 550, 600, 650, 700, 750, 800, 850, 900, 950, 1000, 1200, 1400, 1600, 1800, 1900, 2000, 3000, 4000, 5000, 6000, 7000, 8000, 9000, 10000, 15000, or 20000 ng/kg body weight of the subject. In some embodiments, a maximal dose level of a compound for achieving therapy in the disclosed methods of treatment may not exceed about 10, 20, 30, 40, 50, 60, 70, 80, 90, 100, 150, 200, 250, 300, 350, 400, 450, 500, 550, 600, 650, 700, 750, 800, 850, 900, 950, 1000, 1200, 1400, 1600, 1800, 1900, 2000, 3000, 4000, 5000, 6000, 7000, 8000, 9000, 10000, 15000, or 20000 ng/kg body weight of the subject. Minimal and/or maximal dose levels of the compounds for achieving therapy in the disclosed methods of treatment may include dose levels falling within ranges having as end-points any of these disclosed dose levels (e.g., 500-2000 ng/kg body weight of the subject).

As used herein the term “effective amount” refers to the amount or dose of the compound, upon single or multiple dose administration to the subject, which provides the desired effect in the subject under diagnosis or treatment. The disclosed methods may include administering an effective amount of the disclosed compounds (e.g., as present in a pharmaceutical composition) for treating a disease or disorder associated with protein kinase, e.g., RIPK2, and/or NOD2 activity.

An effective amount can be readily determined by the attending diagnostician, as one skilled in the art, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

A typical daily dose may contain from about 0.01 mg/kg to about 100 mg/kg (such as from about 0.05 mg/kg to about 50 mg/kg and/or from about 0.1 mg/kg to about 25 mg/kg) of each compound used in the present method of treatment.

Compositions can be formulated in a unit dosage form, each dosage containing from about 1 to about 500 mg of each compound individually or in a single unit dosage form, such as from about 5 to about 300 mg, from about 10 to about 100 mg, and/or about 25 mg. The term “unit dosage form” refers to a physically discrete unit suitable as unitary dosages for a patient, each unit containing a predetermined quantity of active material calculated to produce the desired therapeutic effect, in association with a suitable pharmaceutical carrier, diluent, or excipient.

Oral administration is an illustrative route of administering the compounds employed in the compositions and methods disclosed herein. Other illustrative routes of administration include transdermal, percutaneous, subcutaneous, intravenous, intraperitoneal, intramuscular, intranasal, buccal, intrathecal, intracerebral, or intrarectal routes. The route of administration may be varied in any way, limited by the physical properties of the compounds being employed and the convenience of the subject and the caregiver.

The compounds and compositions disclosed herein may be administered in methods of treatment as known in the art. Accordingly, various such compounds and compositions can be administered in conjunction with such a method in any suitable way. For example, administration may comprise oral, intravenous, intraarterial, intramuscular, subcutaneous, intraperitoneal, parenteral, transdermal, intravaginal, intranasal, mucosal, sublingual, topical, rectal or subcutaneous administration, or any combination thereof.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Example 1: Preparation of Compounds

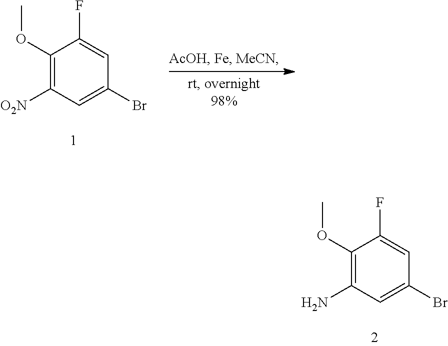

### 5-bromo-3-fluoro-2-methoxyaniline (2)

### (Method A)

A solution of 1 (100 mg, 0.4 mmol), acetonitrile (1 ml), and glacial (0.36 ml, 6 mmol) was stirred in an ice bath for ten minutes. Iron powder (156.4 mg, 2.8 mmol) was added to the mixture in small portions. The reaction was stirred at room temperature overnight. The reaction mixture was filtered through celite and diluted with ethyl acetate which was then neutralized with 3N sodium hydroxide. The organic phase was separated, and the aqueous layer was washed twice more with ethyl acetate. The combined organic layers were washed with water and brine, dried over anhydrous Na₂SO₄, and concentrated. Product 2 (98%) was afforded as a brown oil. ¹H-NMR (600 MHz, CDCl₃) δ 6.62 (d, J=7.9 Hz, 2H), 3.81-4.11 (m, 5H). ¹³C-NMR (150 MHz, CDCl₃) δ 155.5 (d, J_{CF}=246 Hz), 142.08 (d, J_{CF}=6 Hz), 133.75 (d, J_{CF}=13.5 Hz), 115.33 (d, J_{CF}=12 Hz), 113.63 (d, J_{CF}=3 Hz), 109.3 (d, J_{CF}=22.5 Hz), 60.73 (d, J_{CF}=4.5 Hz).

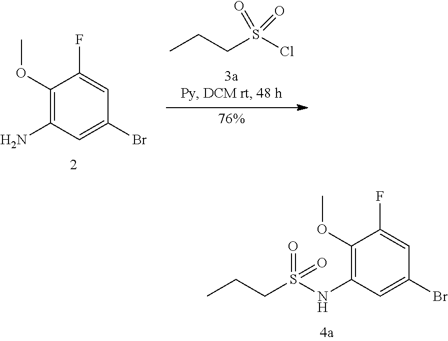

### N-(5-Bromo-3-fluoro-2-methoxyphenyl)propane-1-sulfonamide (4a)

### (Method B)

To a solution of 2 (189 mg, 0.85 mmol) and anhydrous DCM (8.6 ml) was added 3a (0.1 ml, 0.93 mmol) and pyridine (0.14 ml, 1.7 mmol). The reaction was stirred at room temperature for 48 h. The crude product was separated by chromatography (10-15% EtOAc in hexane), and yielded 4a (76%) as a yellow solid. ¹H-NMR (600 MHz, CDCl₃) δ 7.49 (s, 1H), 7.03 (dd, J=11.0, 2.1 Hz, 1H), 6.95 (d, J=6.9 Hz, 1H), 4.00 (d, J=2.1 Hz, 3H), 3.09-3.12 (m, 2H), 1.85 (m, 2H), 1.05 (t, J=7.4 Hz, 3H). ¹³C-NMR (150 MHz, CDCl₃) δ 154.31 (d, J_{CF}=252.17 Hz), 135.61, 132.15, 116.67, 115.71 (d, J_{CF}=21.14 Hz), 115.45 (d, J_{CF}=10.57 Hz), 61.6 (d, J_{CF}=7.55 Hz), 53.70, 17.22, 12.8.

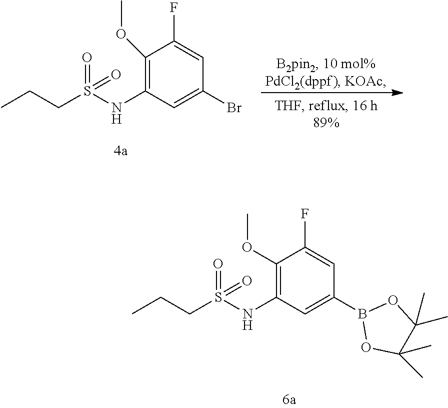 [SMILES: CCCS(=O)(=O)Nc1cc(Br)cc(F)c1OC ; CCCS(=O)(=O)Nc1cc(B2OC(C)(C)C(C)(C)O2)cc(F)c1OC]

### N-(3-Fluoro-2-methoxy-5-(4,4,5,5-tetramethyl-1,3,2-dioxaborolan-2-yl)phenyl)propane-1-sulfonamide (6a)

### (Method C)

A mixture of 4a (40 mg, 0.123 mmol), B₂Pin₂ (38.9 mg, 0.153 mmol), PdCl₂(dppf) (9 mg, 0.012), and KOAc (36.94 mg, 0.38 mmol) in anhydrous THF (1.15 ml) was refluxed for 16 h under argon. The reaction was quenched with water and the aqueous layer was extracted twice with EtOAc. The combined organic phases were washed with brine, dried over anhydrous Na₂SO₄, and concentrated. The crude product was separated by chromatography (5-15% EtOAc in hexane), and yielded 6a (89%) as a white solid. ¹H-NMR (600 MHz, CDCl₃) δ 7.68 (s, 1H), 7.30 (d, J=12.1 Hz, 1H), 6.87 (s, 1H), 4.04 (s, 3H), 3.10 (t, J=7.9 Hz, 2H), 1.85 (m, 2H), 1.32 (s, 12H), 1.03 (t, J=7.6 Hz, 3H). ¹³C-NMR (150 MHz, CDCl₃) δ 154.32 (d, J_{CF}=245.5 Hz), 139.2 (d, J_{CF}=245.5 Hz), 130.39, 120.54, 118.7 (d, J_{CF}=17.25 Hz), 84.18, 61.43 (d, J_{CF}=8.55 Hz), 53.36, 24.80, 17.22, 12.81.

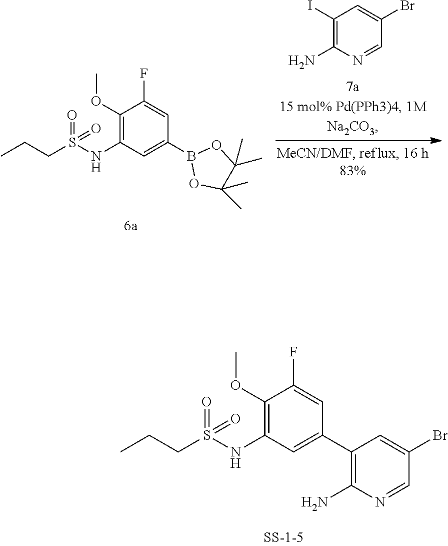 [SMILES: Nc1ncc(Br)cc1I ; CCCS(=O)(=O)Nc1cc(B2OC(C)(C)C(C)(C)O2)cc(F)c1OC ; CCCS(=O)(=O)Nc1cc(-c2cc(Br)cnc2N)cc(F)c1OC]

### N-(5-(2-amino-5-bromopyridin-3-yl)-3-fluoro-2-methoxyphenyl)propane-1-sulfonamide (SS-1-5)

### (Method D)

A mixture of 6a (22.6 mg, 0.06 mmol), 7a (18.1 mg, 0.06 mmol), Pd(PPh₃)₄ (1.05 mg, 0.006 mmol), and 1M Na₂CO₃ (0.14 ml 0.14 mmol) in anhydrous MeCN (1 ml) and anhydrous DMF (0.5 ml) under argon was stirred at 90° C. for 16 h. The reaction was quenched with water and the aqueous layer was extracted twice with EtOAc. The combined organic phases were washed with brine, dried over anhydrous Na₂SO₄, and concentrated. The crude product was separated by chromatography (10-30% EtOAc in hexane), and yielded SS-1-5 (83%) as a white solid. mp 162-163° C. ¹H-NMR (600 MHz, CDCl₃) δ 8.11 (s, 1H), 7.45 (d, J=1.7 Hz, 1H), 7.38 (s, 1H), 7.03 (s, 1H), 6.96 (d, J=12.1 Hz, 1H), 4.68 (s, 2H), 4.07 (d, J=2.1 Hz, 3H), 3.12 (t, J=7.7 Hz, 2H), 1.89 (m, 2H), 1.06 (t, J=7.4 Hz, 3H). ¹³C-NMR (150 MHz, CDCl₃) δ 154.5 (d, J_{CF}=247.5 Hz), 154.34, 148.27, 139.69, 136.5 (d, J_{CF}=11.4 Hz), 132.23 (d, J_{CF}=8.5 Hz), 131.74 (d, J_{CF}=5.7 Hz), 121.25, 114.26, 112.65 (d, J_{CF}=21.1 Hz), 108.39, 61.63, 61.6 (d, J_{CF}=7 Hz), 54.20, 17.25, 12.84. HRMS (ESI) m/z: [M+H]⁺ calculated for C₁₅H₁₇BrFN₃O₃S: 418.0231; observed 418.0235. Purity 98%, t_{R}=18.96 min.

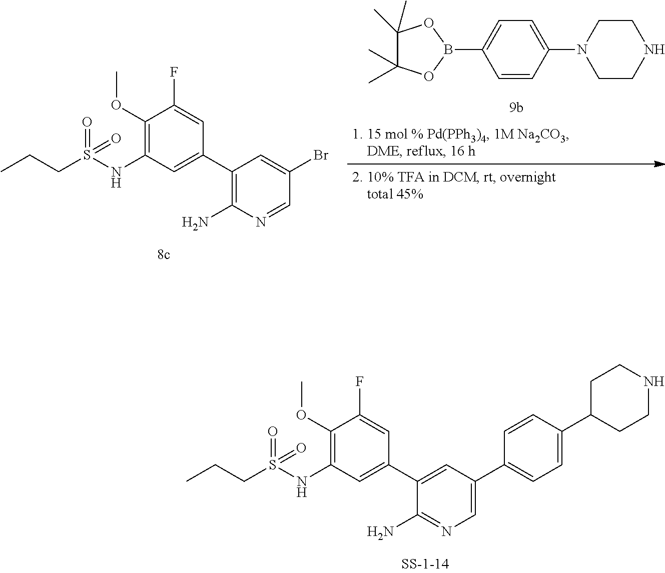 [SMILES: CCCS(=O)(=O)Nc1cc(-c2cc(Br)cnc2N)cc(F)c1OC ; CC1(C)OB(c2ccc(N3CCNCC3)cc2)OC1(C)C ; CCCS(=O)(=O)Nc1cc(-c2cc(-c3ccc(C4CCNCC4)cc3)cnc2N)cc(F)c1OC]

### N-(5-(2-amino-5-(4-(piperidin-4-yl)phenyl)pyridin-3-yl)-3-fluoro-2-methoxyphenyl)propane-1-sulfonamide (SS-1-14)

### (Method E)

To a mixture of 8c (21.4 mg, 0.051 mmol), 9b (22.6 mg, 0.058 mmol), Pd(PPh₃)₄ (9.01 mg, 0.008 mmol), and 1M Na₂CO₃ (0.11 ml, 0.11 mmol) in anhydrous DME (1 ml) under argon was stirred at 90° C. for 16 h. The reaction was quenched with water and the aqueous layer was extracted twice with EtOAc. The combined organic phases were washed with brine, dried over anhydrous Na₂SO₄, and concentrated. The crude product was separated by chromatography (10-50% EtOAc in hexane). The coupling product was dissolved in anhydrous DCM (2 ml) and TFA (0.2 ml) and stirred at room temperature overnight. The reaction mixture was quenched with saturated the aqueous Na₂HCO₃ and extracted with DCM. The crude product was separated by chromatography (10% MeOH is DCM), and yielded SS-1-14 (45%) as a white solid. mp 90-92° C. ¹H-NMR (600 MHz, CDCl₃) δ 8.29 (s, 1H), 7.54 (d, J=2.1 Hz, 1H), 7.47 (d, J=7.9 Hz, 2H), 7.44 (s, 1H), 7.31 (d, J=8.3 Hz, 2H), 7.03 (dd, J=11.9, 1.5 Hz, 1H), 4.82 (s, 2H), 4.08 (d, J=1.7 Hz, 3H), 3.66 (d, J=12.1 Hz, 2H), 3.12-3.15 (m, 2H), 3.06 (t, J=12.1 Hz, 2H), 2.82 (t, J=12.1 Hz, 1H), 2.27 (q, J=12.1 Hz, 2H), 2.07 (d, J=13.4 Hz, 2H), 1.89 (m, 2H), 1.06 (t, J=7.4 Hz, 3H). ¹³C-NMR (150 MHz, CDCl₃) δ 154.82, 154.6 (d, J_{CF}=252.17 Hz), 145.76, 142.40, 136.62, 136.55, 136.47, 136.35, 133.4 (d, J_{CF}=7.2 Hz), 131.66 (d, J_{CF}=5.7 Hz), 127.32, 126.61, 119.70, 114.86, 112.8 (d, J_{CF}=18.6 Hz), 61.6 (d, J_{CF}=7.2 Hz), 54.16, 44.46, 40.21, 29.86, 17.23, 12.84. HRMS (ESI) m/z: [M+H]⁺ calculated for C₂₆H₃₁FN₄O₃S: 499.2174; observed 499.2181. Purity 97%, t_{R}=16.8 min.

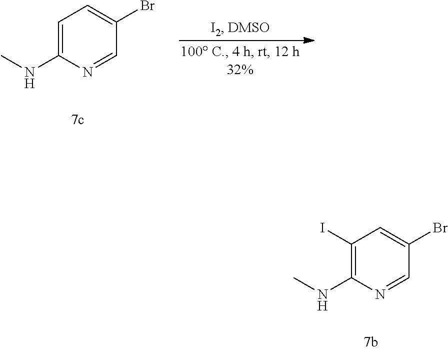

### 5-bromo-3-iodo-N-methylpyridin-2-amine (7b)

To a solution of 7c (120 mg, 0.17 mmol) and DMSO (3 ml) was added iodine (302 mg, 0.24 mmol). The mixture was stirred at 100° C. for 4 h and then 12 h at room temperature.² The reaction was quenched with saturated Na₂S₂O₅ the aqueous solution and the aqueous layer was extracted with EtOAc. The combined organic phases were washed with brine, dried over anhydrous Na₂SO₄, and concentrated. The crude product was separated by chromatography (5-10% EtOAc in hexane), and yielded 7b (32%) as a white solid. ¹H-NMR (600 MHz, CDCl₃) δ 8.12 (d, J=2.1 Hz, 1H), 7.90 (d, J=2.1 Hz, 1H), 4.98 (s, 1H), 2.98 (d, J=4.8 Hz, 3H). ¹³C-NMR (150 MHz, CDCl₃) δ 155.71, 148.04, 147.35, 105.70, 79.67, 29.45.

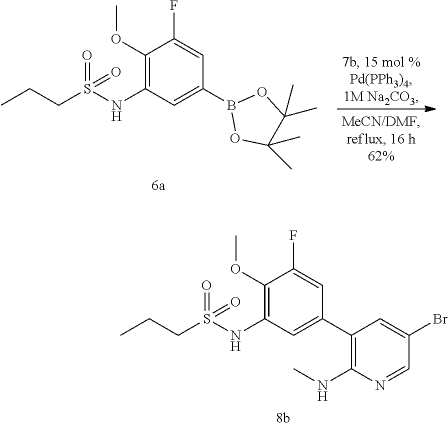

### N-(5-(5-bromo-2-(methylamino)pyridin-3-yl)-3-fluoro-2-methoxyphenyl)propane-1-sulfonamide (8b)

Method C; The crude product was separated by chromatography (30-70% EtOAc in hexane), and yielded 8b (62%) as an off white solid. ¹H-NMR (600 MHz, CDCl₃) δ 8.18 (d, J=2.4 Hz, 1H), 7.33 (d, J=2.4 Hz, 1H), 7.32 (t, J=1.5 Hz, 1H), 6.98 (s, 1H), 6.90 (dd, J=12.1, 2.1 Hz, 1H), 4.64 (d, J=4.8 Hz, 1H), 4.07 (d, J=2.8 Hz, 3H), 3.10-3.13 (m, 2H), 2.93 (d, J=4.8 Hz, 3H), 2.18 (s, 2H), 1.85-1.92 (m, 2H), 1.06 (t, J=7.4 Hz, 3H). ¹³C-NMR (150 MHz, CDCl₃) δ 154.65, 154.57 (d, J_{CF}=247 Hz), 147.98, 138.70, 136.4 (d, J_{CF}=12 Hz), 132.12 (d, J_{CF}=9 Hz), 131.8 (d, J_{CF}=4.35 Hz), 121.75, 114.37, 112.82 (d, J_{CF}=19.5 Hz), 106.55, 61.6 (d, J_{CF}=6 Hz), 54.15, 28.72, 17.23, 12.83.

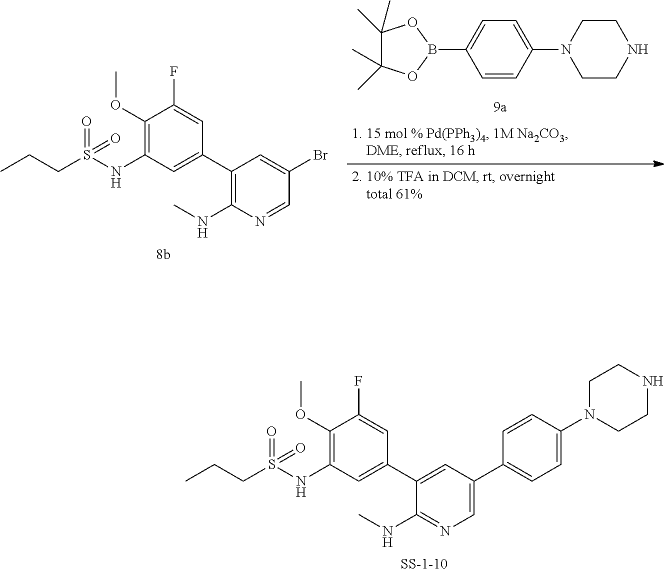 [SMILES: CCCS(=O)(=O)Nc1cc(-c2cc(Br)cnc2NC)cc(F)c1OC ; CC1(C)OB(c2ccc(N3CCNCC3)cc2)OC1(C)C ; CCCS(=O)(=O)Nc1cc(-c2cc(-c3ccc(N4CCNCC4)cc3)cnc2NC)cc(F)c1OC]

### N-(3-fluoro-2-methoxy-5-(2-(methylamino)-5-(4-(piperazin-1-yl)phenyl)pyridin-3-yl)phenyl)propane-1-sulfonamide (SS-1-10)

Method E; The crude product was separated by chromatography (10% MeOH in DCM), and yielded SS-1-10 (61%) as a beige solid. mp 166-168° C. ¹H-NMR (600 MHz, CDCl₃) δ 8.39 (d, J=2.1 Hz, 1H), 7.43-7.45 (m, 3H), 7.38 (s, 1H), 6.96-7.00 (m, 3H), 4.64 (q, J=4.7 Hz, 1H), 4.07 (d, J=2.1 Hz, 3H), 3.24 (s, 4H), 3.11-3.14 (m, 6H), 3.01 (d, J=4.8 Hz, 3H), 1.87-1.92 (m, 2H), 1.06 (t, J=7.4 Hz, 3H). ¹³C-NMR (150 MHz, CD₃OD) δ 157.8 (d, J_{CF}=245.7 Hz), 156.52, 151.51, 144.91, 140.14 (d, J_{CF}=11.55 Hz), 136.70, 134.5 (d, J_{CF}=7.5 Hz), 133.57, 131.23, 127.65, 126.54, 122.68, 119.78, 118.10, 114.65 (d, J_{CF}=19.5 Hz), 62.14, 54.99, 45.81, 29.06, 18.34, 13.17. HRMS (ESI) m/z: [M+H]⁺ calculated for C₂₆H₃₂FN₅O₃S: 514.2283; observed 4514.2290. Purity 95.5%, t_{R}=16.74 min.

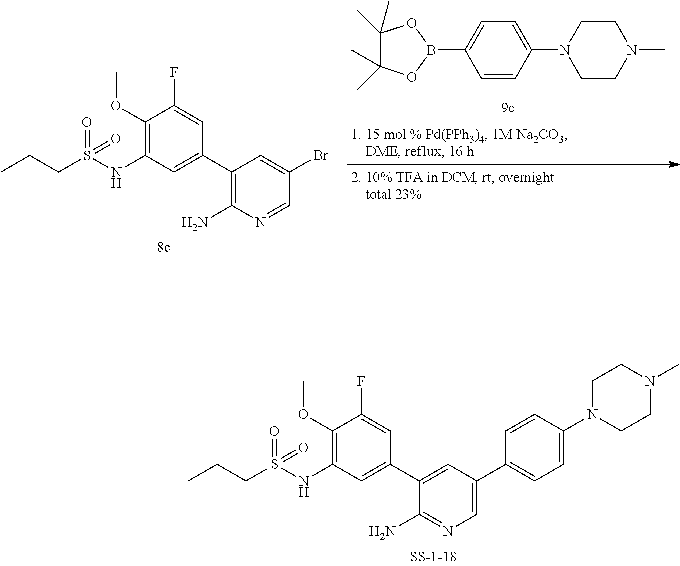

### N-(5-(2-amino-5-(4-(4-methylpiperazin-1-yl)phenyl)pyridin-3-yl)-3-fluoro-2-methoxyphenyl)propane-1-sulfonamide (SS-1-18)

### (Method F)

To a mixture of 8c (15 mg, 0.03 mmol), 9c (10 mg, 0.033 mmol), Pd(PPh₃)₄ (9 mg, 0.004 mmol), and 1M Na₂CO₃ (0.06 ml 0.06 mmol) in anhydrous DME (1 ml) under argon was stirred at 90° C. for 16 h. The reaction was quenched with water and the aqueous layer was extracted twice with DCM. The combined organic phases were washed with brine, dried over anhydrous Na₂SO₄, and concentrated. The crude product was separated by chromatography (5% MeOH in DCM), and yielded SS-1-18 (23%) as a white solid. mp 184-188° C. ¹H-NMR (600 MHz, CDCl₃) δ 8.29 (s, 1H), 7.53 (d, J=1.4 Hz, 1H), 7.44 (s, 2H), 7.43 (s, 1H), 7.02-7.04 (m, 1H), 6.99 (d, J=8.3 Hz, 2H), 4.67 (s, 2H), 4.07 (d, J=1.7 Hz, 3H), 3.26 (t, J=4.6 Hz, 4H), 3.11-3.14 (m, 2H), 2.61 (t, J=4.6 Hz, 4H), 2.37 (s, 3H), 1.89 (m, 2H), 1.05 (t, J=7.4 Hz, 3H). ¹³C-NMR (150 MHz, CDCl₃) δ 154.6 (d, J_{CF}=247 Hz), 154.14, 150.37, 145.44, 136.26 (d, J_{CF}=12.9 Hz), 135.96, 133.76 (d, J_{CF}=9 Hz), 131.61 (d, J_{CF}=4.35 Hz), 128.89, 127.87, 126.94, 119.60, 116.25, 114.60, 112.6 (d, J_{CF}=19.5 Hz), 61.58 (d, J_{CF}=7.2 Hz), 55.00, 54.15, 48.87, 46.11, 17.23, 12.84. HRMS (ESI) m/z: [M+H]⁺ calculated for C₂₆H₃₂FN₅O₃S: 514.2283; observed 514.2293. Purity 95.4%, t_{R}=16.64 min.

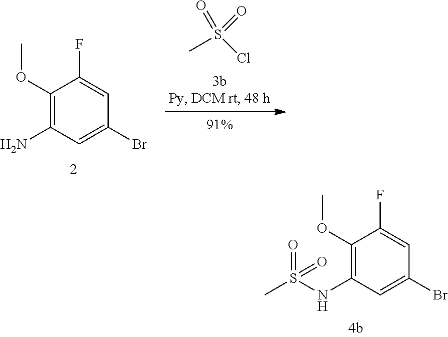

### N-(5-bromo-3-fluoro-2-methoxyphenyl)methanesulfonamide (4b)

Method B; The crude product was separated by chromatography (20% EtOAc in Hexane), and yielded 4b (91%) as a white solid. ¹H-NMR (600 MHz, CDCl₃) δ 7.49 (t, J=1.7 Hz, 1H), 7.05 (dd, J=10.8, 2.2 Hz, 1H), 7.00 (d, J=14.1 Hz, 1H), 4.00 (d, J=2.4 Hz, 3H), 3.06 (s, 3H). ¹³C-NMR (150 MHz, CDCl₃) δ 155.3 (d, J_{CF}=245 Hz), 136.05 (d, J_{CF}=12.9 Hz), 131.94 (d, J_{CF}=6 Hz), 117.18, 116.07 (d, J_{CF}=21 Hz), 115.4 (d, J_{CF}=9.81 Hz), 61.6 (d, J_{CF}=7.2 Hz), 39.82.

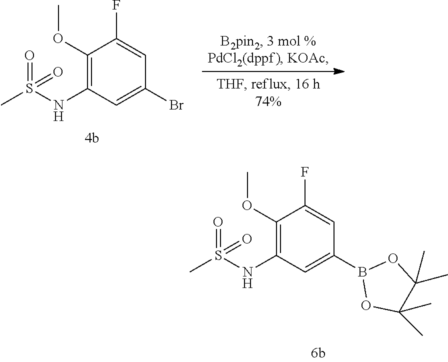

### N-(3-fluoro-2-methoxy-5-(4,4,5,5-tetramethyl-1,3,2-dioxaborolan-2-yl)phenyl)methanesulfonamide (6b)

Method C; The crude product was separated by chromatography (10-15% EtOAc in Hexane), and yielded 6b (74%) as a white solid. ¹H-NMR (600 MHz, CDCl₃) δ 7.68 (s, 1H), 7.33 (d, J=11.7 Hz, 1H), 7.00 (s, 1H), 4.05 (d, J=2.4 Hz, 3H), 3.05 (s, 3H), 1.31 (s, 12H). ¹³C-NMR (150 MHz, CDCl₃) δ 153.81 (d, J_{CF}=245.5 Hz), 139.72 (d, J_{CF}=13 Hz), 130.16 (d, J_{CF}=3 Hz), 121.30, 119.15 (d, J_{CF}=18 Hz), 84.16, 74.97, 61.42 (d, J_{CF}=9 Hz), 39.63, 24.76, 24.50.

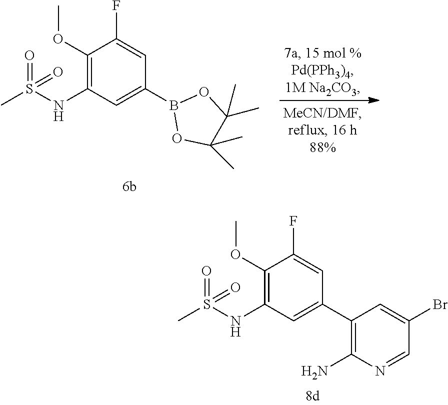

### N-(5-(2-amino-5-bromopyridin-3-yl)-3-fluoro-2-methoxyphenyl)methanesulfonamide (8d)

Method D; The crude product was separated by chromatography (20-30% EtOAc in Hexane), and yielded 8d (88%) as a white solid. ¹H-NMR (600 MHz, CDCl₃) δ 8.12 (d, J=2.1 Hz, 1H), 7.45 (d, J=2.4 Hz, 1H), 7.38 (s, 1H), 7.02 (s, 1H), 6.99 (dd, J=12.1, 1.7 Hz, 1H), 4.64 (s, 2H), 4.08 (d, J=2.4 Hz, 3H), 3.08 (s, 3H). ¹³C-NMR (150 MHz, CDCl₃) δ 154.5 (d, J_{CF}=246 Hz), 154.28, 148.35, 139.71, 136.75 (d, J_{CF}=12 Hz), 132.3 (d, J_{CF}=7.5 Hz), 131.5 (d, J_{CF}=5.7 Hz), 121.16, 114.68, 113.02 (d, J_{CF}=20.1 Hz), 108.45, 61.3 (d, J_{CF}=7.5 Hz), 40.24.

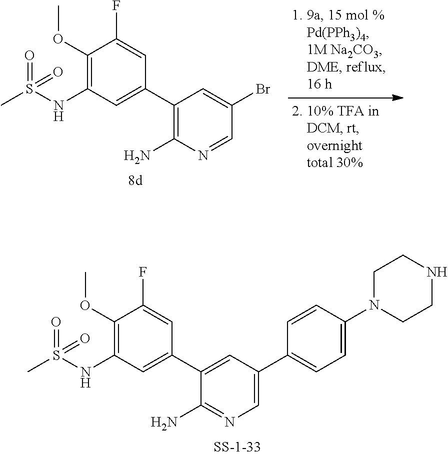 [SMILES: COc1c(F)cc(-c2cc(Br)cnc2N)cc1NS(C)(=O)=O ; COc1c(F)cc(-c2cc(-c3ccc(N4CCNCC4)cc3)cnc2N)cc1NS(C)(=O)=O]

### N-(5-(2-amino-5-(4-(piperazin-1-yl)phenyl)pyridin-3-yl)-3-fluoro-2-methoxyphenyl)methanesulfonamide (SS-1-33)

Method E; The crude product was separated by chromatography (5% MeOH in DCM), and yielded SS-1-33 (30%) as a light yellow solid. 164-166 mp ° C. ¹H-NMR (600 MHz, CD₃OD) S 8.15 (s, 1H), 7.61 (d, J=2.0 Hz, 1H), 7.49 (d, J=8.5 Hz, 2H), 7.39 (s, 1H), 7.14 (dd, J=12.3, 1.8 Hz, 1H), 7.06 (d, J=9.0 Hz, 2H), 4.61 (s, 1H), 4.01 (d, J=1.3 Hz, 3H), 3.36 (d, J=5.5 Hz, 4H), 3.05 (s, 3H). ¹³C-NMR (150 MHz, CD₃OD) δ 157.82 (d, J_{CF}=244.8 Hz), 156.44, 156.01, 151.00, 145.21, 141.78, 140.48, 137.60, 134.61, 133.47, 131.54, 128.26, 127.93, 119.97, 118.35, 114.82, 114.69, 62.16 (d, J_{CF}=7.5 Hz), 48.27, 45.06, 40.31. HRMS (ESI) m/z: [M+H]⁺ calculated for C₂₃H₂₆FN₅O₃S: 472.1813; observed 472.1819. Purity 97%, t_{R}=15.72 min.

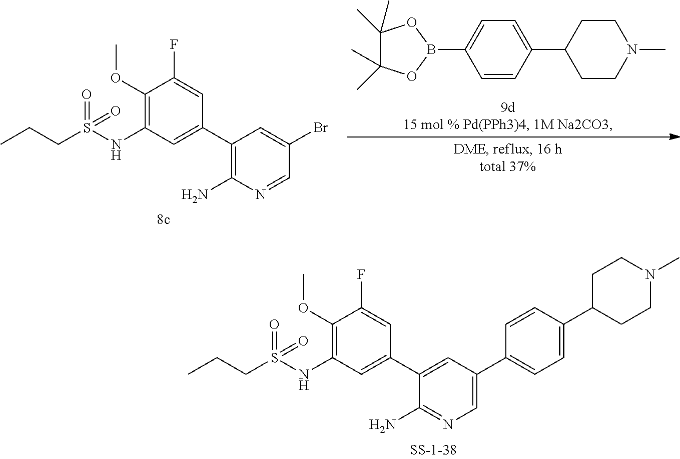

### N-(5-(2-amino-5-(4-(1-methylpiperidin-4-yl)phenyl)pyridin-3-yl)-3-fluoro-2-methoxyphenyl)propane-1-sulfonamide (SS-1-38)

Method F; The crude product was separated by chromatography (10% MeOH in DCM), and yielded SS-1-38 (37%) as a yellow solid. mp 143-145° C. ¹H-NMR (600 MHz, CDCl₃) δ 8.31 (s, 1H), 7.55 (d, J=2.1 Hz, 1H), 7.45-7.47 (m, 3H), 7.30 (d, J=7.9 Hz, 2H), 7.03 (dd, J=11.7, 1.4 Hz, 1H), 4.75 (s, 2H), 4.07 (d, J=2.1 Hz, 3H), 3.10-3.14 (m, 4H), 2.53-2.58 (m, 1H), 2.42 (s, 3H), 2.21 (t, J=11.2 Hz, 2H), 1.86-1.99 (m, 6H), 1.06 (t, J=7.4 Hz, 3H). ¹³C-NMR (150 MHz, CDCl₃) δ 154.79, 154.75 (d, J_{CF}=247 Hz), 145.98, 144.79, 136.59, 136.49, 136.02, 133.69 (d, J_{CF}=7.2 Hz), 131.8 (d, J_{CF}=5.7 Hz), 127.88, 127.58, 126.51, 119.75, 114.89, 112.92 (d, J_{CF}=18.6 Hz), 61.72 (d, J_{CF} 7.2 Hz), 56.18, 54.30, 46.08, 41.40, 32.98, 17.37, 12.97. HRMS (ESI) m/z: [M+H]⁺ calculated for C₂₇H₃₃FN₄O₃: 513.2330; observed 513.2334. Purity 95%, t_{R}=18.2 min.

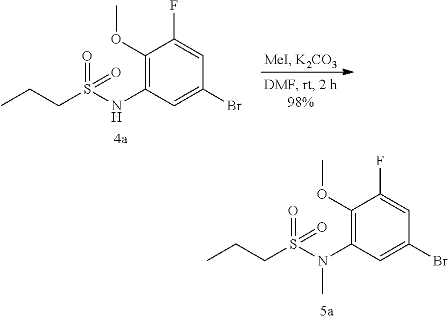 [SMILES: CCCS(=O)(=O)Nc1cc(Br)cc(F)c1OC ; CCCS(=O)(=O)N(C)c1cc(Br)cc(F)c1OC]

### N-(5-bromo-3-fluoro-2-methoxyphenyl)-N-methylpropane-1-sulfonamide (5a)

### (Method G)

To a mixture of 4a (50.3 mg, 0.154 mmol) is anhydrous DMF (0.5 ml) was added K₂CO₃ (64 mg, 0.47 mmol) at room temperature under argon. After 10 minutes methyl iodide (0.019 ml, 0.31 mmol) was added dropwise and stirred at room temperature for 2 h. The reaction was quenched with water and the aqueous layer was extracted with EtOAc. The combined organic phases were washed with brine, dried over anhydrous Na₂SO₄, and concentrated. The crude product was separated by chromatography (15% EtOAc in hexane), and yielded 5a (98%) as an off white solid. ¹H-NMR (600 MHz, CDCl₃) δ 7.28 (d, J=1.4 Hz, 1H), 7.26 (dd, J=11.2, 1.9 Hz, 1H), 4.04 (d, J=2.1 Hz, 3H), 3.24 (s, 3H), 3.06-3.09 (m, 2H), 1.92 (m, 2H), 1.08 (t, J=7.4 Hz, 3H) 1.89-1.95 (m, 2H), 1.08 (t, J=7.4 Hz, 3H). ¹³C-NMR (150 MHz, CDCl₃) δ 155.21 (d, J_{CF}=252 Hz), 144.7 (d, J_{CF}=9 Hz), 135.3 (d, J_{CF}=4.5 Hz), 129.63 (d, J_{CF}=3 Hz), 120.66 (d, J_{CF}=24 Hz), 113.77 (d, J_{CF}=10.5 Hz), 61.33 (d, J_{CF}=6 Hz), 53.53, 38.50, 17.06, 13.09.

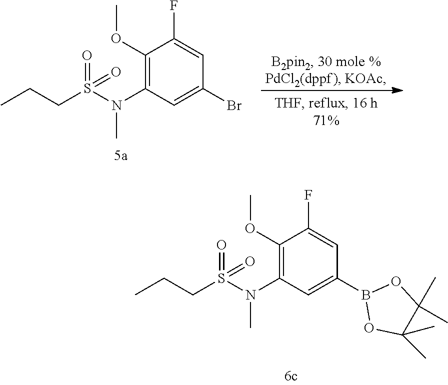 [SMILES: CCCS(=O)(=O)N(C)c1cc(Br)cc(F)c1OC ; CCCS(=O)(=O)N(C)c1cc(B2OC(C)(C)C(C)(C)O2)cc(F)c1OC]

### N-(3-fluoro-2-methoxy-5-(4,4,5,5-tetramethyl-1,3,2-dioxaborolan-2-yl)phenyl)-N-methylpropane-1-sulfonamide (6c)

Method C; The crude product was separated by chromatography (5-10% EtOAc in hexane), and yielded 6c (71%) as a yellow oil. ¹H-NMR (600 MHz, CDCl₃) δ 7.54 (s, 1H), 7.50 (d, J=12.1 Hz, 1H), 4.08 (d, J=3.1 Hz, 3H), 3.24 (s, 3H), 3.07-3.10 (m, 2H), 1.90-1.96 (m, 2H), 1.33 (s, 12H), 1.08 (t, J=7.4 Hz, 3H). ¹³C-NMR (150 MHz, CDCl₃) δ 154.72 (d, J_{CF}=249 Hz), 147.77 (d, J_{CF}=9 Hz), 133.77, 132.82, 123.14 (d, J_{CF}=18 Hz), 84.20, 61.2 (d, J_{CF}=9 Hz), 53.59, 38.55, 24.84, 17.13, 13.17.

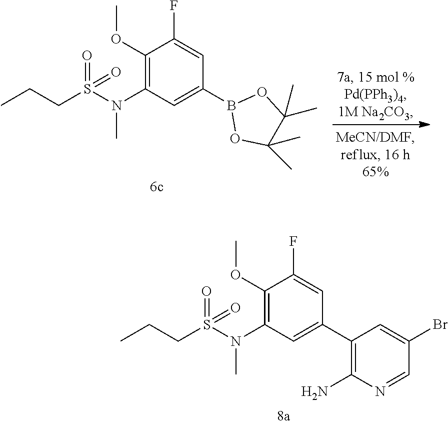

### N-(5-(2-amino-5-bromopyridin-3-yl)-3-fluoro-2-methoxyphenyl)-N-methylpropane-1-sulfonamide (8a)

Method D; The crude product was separated by chromatography (15-25% EtOAc in Hexane), and yielded 8a (65%) as a white solid. ¹H-NMR (600 MHz, CDCl₃) δ 8.10 (d, J=2.1 Hz, 1H), 7.45 (d, J=2.1 Hz, 1H), 7.28 (s, 1H), 7.18 (dd, J=12.4, 1.7 Hz, 1H), 4.74 (s, 2H), 4.12 (d, J=2.8 Hz, 3H), 3.31 (s, 3H), 3.06-3.09 (m, 2H), 1.89-1.96 (m, 2H), 1.08 (t, J=7.4 Hz, 3H). ¹³C-NMR (150 MHz, CDCl₃) δ 154.43 (d, J_{CF}=268.5 Hz), 154.53, 148.39, 144.84 (d, J_{CF}=10.5 Hz), 139.64, 134.56, 131.31, 127.07, 120.77, 117.56 (d, J_{CF}=20.1 Hz), 108.42, 61.43 (d, J_{CF}=8.7 Hz), 53.56, 38.42, 17.20, 13.23.

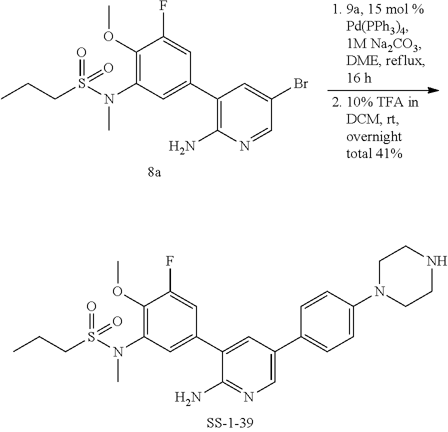 [SMILES: CCCS(=O)(=O)N(C)c1cc(-c2cc(Br)cnc2N)cc(F)c1OC ; CCCS(=O)(=O)N(C)c1cc(-c2cc(-c3ccc(N4CCNCC4)cc3)cnc2N)cc(F)c1OC]

### N-(5-(2-amino-5-(4-(piperazin-1-yl)phenyl)pyridin-3-yl)-3-fluoro-2-methoxyphenyl)-N-methylpropane-1-sulfonamide (SS-1-39)

Method E; The crude product was separated by chromatography (5% MeOH in DCM), and yielded SS-1-39 (41%) as a yellow solid. mp 74-76° C. ¹H-NMR (600 MHz, CD₃OD) δ 8.15 (d, J=2.4 Hz, 1H), 7.61 (d, J=2.4 Hz, 1H), 7.45 (d, J=8.6 Hz, 2H), 7.30-7.35 (dd, J=12, J=2.4, 2H), 7.01 (d, J=8.6 Hz, 2H), 4.09 (d, J=2.4 Hz, 3H), 3.29 (s, 3H), 3.18-3.21 (m, 6H), 3.04 (t, J=5.0 Hz, 4H), 1.88 (m, 2H), 1.07 (t, J=7.4 Hz, 3H). ¹³C-NMR (150 MHz, CD₃OD) δ 157.64 (d, J_{CF}=245.7 Hz), 156.29, 152.02, 146.13 (d, J_{CF}=9 Hz), 145.24, 137.55, 136.23, 134.18 (d, J_{CF}=8.25 Hz), 130.54, 128.51, 128.05, 127.72, 121.21, 118.64 (d, J_{CF}=21.6 Hz), 117.89, 62.05, 54.19, 50.37, 46.19, 38.86, 18.26, 13.35. HRMS (ESI) m/z: [M+H]⁺ calculated for C₂₆H₃₂FN₅O₃S: 514.2283; observed 514.2290. Purity 95%, t_{R}=18.41 min.

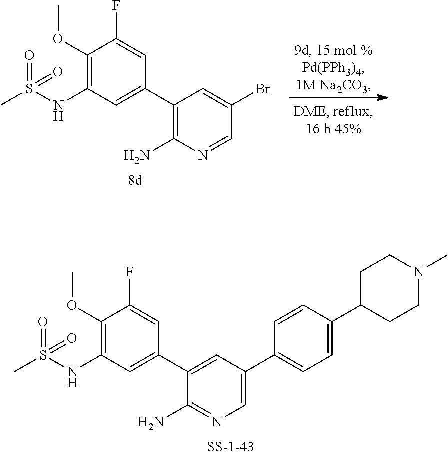

### N-(5-(2-amino-5-(4-(1-methylpiperidin-4-yl)phenyl)pyridin-3-yl)-3-fluoro-2-methoxyphenyl)methanesulfonamide (SS-1-43)

Method F; The crude product was separated by chromatography (10% MeOH in DCM), and yielded SS-1-43 (45%) as a white solid. mp 220-222° C. ¹H-NMR (600 MHz, CDCl₃) δ 8.32 (s, 1H), 7.56 (s, 1H), 7.47 (d, J=7.6 Hz, 2H), 7.45 (s, 1H), 7.30 (d, J=7.9 Hz, 2H), 7.06 (d, J=12.1 Hz, 1H), 4.66 (s, 2H), 4.08 (s, 3H), 3.09 (s, 3H), 3.01 (d, J=11.0 Hz, 2H), 2.52 (td, J=10.3, 5.7 Hz, 1H), 2.35 (s, 3H), 2.06-2.10 (m, 2H), 1.82-1.87 (m, 4H), 1.70 (s, 1H). ¹³C-NMR (150 MHz, CDCl₃) δ 155.6 (d, J_{CF}=245.5 Hz), 154.57, 146.01, 145.34, 136.51, 136.40, 135.73, 133.76, 131.45, 127.99, 127.50, 126.41, 119.52, 114.85, 113.1 (d, J_{CF}=19.5 Hz), 61.6 (d, J_{CF}=7.5 Hz), 56.32, 46.45, 41.67, 40.22, 33.45. HRMS (ESI) m/z: [M+H]⁺ calculated for C₂₅H₂₉FN₄O₃S: 485.2017; observed 485.2019. Purity 98%, t_{R}=16.1 min.

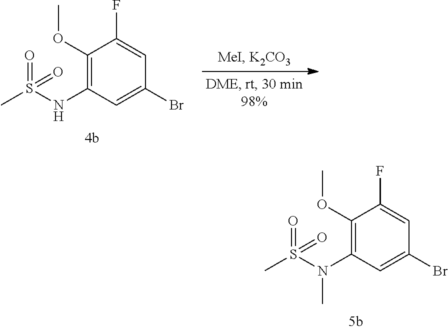 [SMILES: COc1c(F)cc(Br)cc1NS(C)(=O)=O ; COc1c(F)cc(Br)cc1N(C)S(C)(=O)=O]

### N-(5-bromo-3-fluoro-2-methoxyphenyl)-N-methylmethanesulfonamide (5b)

Method G; The crude product was separated by chromatography (5-15% EtOAc in Hexane), and yielded 5b (98%) as a white solid. ¹H-NMR (600 MHz, CDCl₃) δ 7.25-7.29 (m, 2H), 4.05 (d, J=2.8 Hz, 3H), 3.25 (s, 3H), 3.00 (s, 3H). ¹³C-NMR (150 MHz, CDCl₃) δ 155.1 (d, J_{CF}=251.4 Hz), 144.52 (d, J_{CF}=10.05 Hz), 135.04 (d, J_{CF}=5.7 Hz), 129.66 (d, J_{CF}=2.85 Hz), 120.2 (d, J_{CF}=21.6 Hz), 113.85 (d, J_{CF}=11.55 Hz), 61.3 (d, J_{CF}=8.55 Hz), 38.20, 38.15.

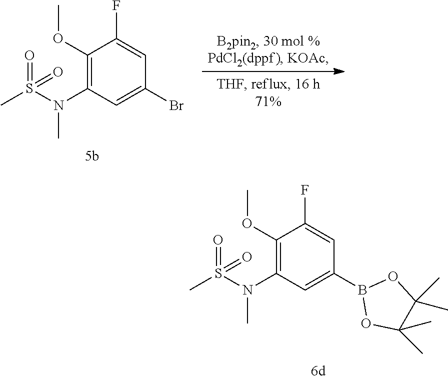

### N-(3-fluoro-2-methoxy-5-(4,4,5,5-tetramethyl-1,3,2-dioxaborolan-2-yl)phenyl)-N-methylmethanesulfonamide (6d)

Method C; The crude product was separated by chromatography (5-15% EtOAc in hexane), and yielded 6d (81%) as a yellow crystal. ¹H-NMR (600 MHz, CDCl₃) δ 7.54 (s, 1H), 7.51 (d, J=12.1 Hz, 1H), 4.09 (d, J=3.1 Hz, 3H), 3.25 (s, 3H), 3.01 (s, 3H), 1.32 (s, 12H). ¹³C-NMR (150 MHz, CDCl₃) δ 154.7 (d, J_{CF}=246 Hz), 147.5 (d, J_{CF}=10.5 Hz), 133.5, 132.7, 123.2 (d, J_{CF}=18 Hz), 84.19, 74.98, 61.2 (d, J_{CF}=9 Hz), 38.22, 38.15, 24.79.

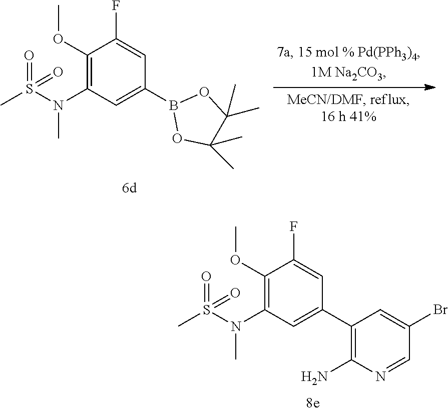

### N-(5-(2-amino-5-bromopyridin-3-yl)-3-fluoro-2-methoxyphenyl)-N-methylmethanesulfonamide (8e)

Method D; The crude product was separated by chromatography (15-25% EtOAc in Hexane), and yielded 8e (41%) as a yellow foam. ¹H-NMR (600 MHz, CDCl₃) δ 8.10 (d, J=2.1 Hz, 1H), 7.45 (d, J=2.1 Hz, 1H), 7.28 (s, 1H), 7.20 (dd, J=12.2, 1.9 Hz, 1H), 4.72 (s, 2H), 4.13 (d, J=2.8 Hz, 3H), 3.31 (s, 3H), 3.00 (s, 3H). ¹³C-NMR (150 MHz, CDCl₃) δ 155.24 (d, J_{CF}=248.4 Hz), 154.39, 148.21, 144.55 (d, J_{CF}=10.5 Hz), 139.52, 134.22 (d, J_{CF}=4.5 Hz), 131.21 (d, J_{CF}=9 Hz), 126.98, 120.58, 117.47 (d, J_{CF}=21.5 Hz), 108.21, 61.27 (d, J_{CF}=8.55 Hz), 38.01, 37.95.

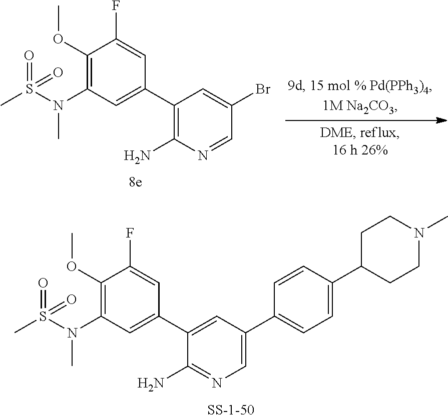

### N-(5-(2-amino-5-(4-(1-methylpiperidin-4-yl)phenyl)pyridin-3-yl)-3-fluoro-2-methoxyphenyl)-N-methylmethanesulfonamide (SS-1-50)

Method F; The crude product was separated by chromatography (10% MeOH in DCM), and yielded SS-1-50 (26%) as yellow solid. mp 152-155° C. ¹H-NMR (600 MHz, CDCl₃) δ 8.31 (s, 1H), 7.56 (d, J=1.7 Hz, 1H), 7.47 (d, J=8.1 Hz, 2H), 7.34 (s, 1H), 7.31 (d, J=8.3 Hz, 2H), 7.25 (s, 1H), 4.72 (s, 2H), 4.13 (d, J=2.8 Hz, 3H), 3.33 (s, 3H), 3.13 (d, J=10.3 Hz, 2H), 3.01 (s, 3H), 2.56-2.60 (m, 1H), 2.45 (s, 3H), 2.25 (t, J=10.8 Hz, 2H), 2.00 (q, J=11.5 Hz, 2H), 1.91 (d, J=12.4 Hz, 2H). ¹³C-NMR (150 MHz, CDCl₃) δ 155.3 (d, J_{CF}=247.5 Hz), 154.70, 145.98, 144.3 (d, J_{CF}=10 Hz), 136.26, 135.95, 134.25, 132.63, 127.79, 127.46, 127.05, 126.42, 118.99, 117.7, 117.56, 61.31 (d, J_{CF}=9 Hz), 56.01, 45.86, 41.21, 38.07, 32.72. HRMS (ESI) m/z: [M+H]⁺ calculated for C₂₆H₃₁FN₄O₃S: 499.2174; observed 499.2180. Purity 97.5%, t_{R}=16.6 min.

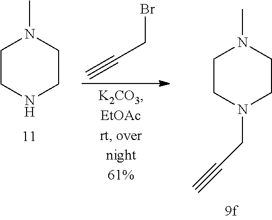

### 1-methyl-4-(prop-2-yn-1-yl)piperazine (9f)

To a mixture of 11 (286.4 mg, 2.86 mmol), K₂CO₃ (790.5 mg, 5.52 mmol) in ethyl acetate was added propargyl bromide (0.22 ml, 2.86 mmol) dropwise and stirred over night at room temperature. DI water was added and stirred for 30 minutes. The organic phase was separated, and the aqueous layer was washed twice more with ethyl acetate. The combined organic layers were washed with water and brine, dried over anhydrous Na₂SO₄, and concentrated. The product 9f (61%) was afforded as a yellow oil. ¹H-NMR (600 MHz, DMSO-D6) δ 3.23 (d, J=1.4 Hz, 2H), 3.15 (s, 1H), 2.19-2.50 (m, 8H), 2.14 (s, 3H). ¹³C-NMR (150 MHz, DMSO-D6) δ 79.45, 75.68, 54.56, 51.06, 45.99, 45.76.

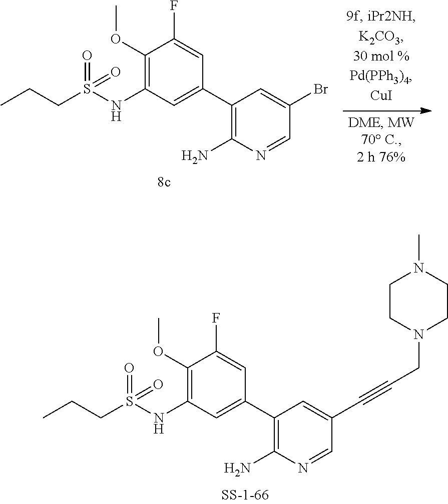 [SMILES: CCCS(=O)(=O)Nc1cc(-c2cc(Br)cnc2N)cc(F)c1OC ; CCCS(=O)(=O)Nc1cc(-c2cc(C#CCN3CCN(C)CC3)cnc2N)cc(F)c1OC]

### N-(5-(2-amino-5-(3-(4-methylpiperazin-1-yl)prop-1-yn-1-yl)pyridin-3-yl)-3-fluoro-2-methoxyphenyl)propane-1-sulfonamide (SS-1-66)

To a mixture of 8c (10 mg, 0.024 mmol), 9f (3.3 m, 0.024 mmol), K₂CO₃ (3.3 mg, 0.024 mmol), CuI (2.3 mg, 0.012 mmol) in dry DME was added Pd(PPh₃)₄ (2.8 mg, 0.0024) and i-Pr2NH and stirred under argan for 10 min. The reaction was placed in MW at 70 C for 2 h. The reaction was dried under rotovate and purified with column chromatography (10% MeOH in DCM) SS-1-66 (76%) was afforded as a off white solid. mp 128-130° C. ¹H-NMR (600 MHz, CDCl₃) δ 8.16 (s, 1H), 7.39 (s, 1H), 7.37 (s, 1H), 6.96 (d, J=12.1 Hz, 1H), 4.81 (s, 2H), 4.07 (s, 3H), 3.51 (s, 2H), 3.13 (t, J=7.7 Hz, 2H), 2.21-2.69 (m, 12H), 2.32 (s, 3H), 1.86-1.92 (m, 2H), 1.06 (t, J=7.4 Hz, 3H). ¹³C-NMR (150 MHz, CDCl₃) δ 155.49 (d, J_{CF}=245.7 Hz), 154.79, 151.07, 140.47, 136.48 (d, J_{CF}=11.4 Hz), 132.83, 131.81, 119.15, 114.51, 112.8 (d, J_{CF}=20.1 Hz), 110.39, 85.35, 82.42, 61.7 (d, J_{CF}=7.2 Hz), 55.06, 54.28, 52.08, 47.83, 46.03, 17.37, 12.96. HRMS (ESI) m/z: [M+H]⁻ calculated for C₂₃H₃₀FN₅O₃S: 476.2126; observed 476.2126. Purity 99%, t_{R}=15.9 min.

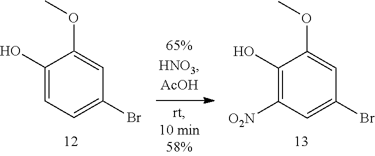

### 4-bromo-2-methoxy-6-nitrophenol (13)

To a solution of 12 (203 mg, 1 mmol) in glacial acetic acid (2 ml) was added 65% HNO₃ dropwise. The mixture was stirred at room temperature for 10 minutes and was poured over ice water. The precipitant was filtered, washed with water, and dried under vacuum. The product 13 (58%) was afforded as a yellow solid. ¹H-NMR (600 MHz, CDCl₃) δ 10.72 (s, 1H), 7.86 (d, J=2.1 Hz, 1H), 7.21 (d, J=1.7 Hz, 1H), 3.96 (s, 3H). ¹³C-NMR (150 MHz, CDCl₃) δ 150.71, 145.69, 134.03, 120.74, 118.10, 110.87, 56.96.

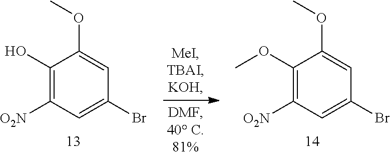

### 5-bromo-1,2-dimethoxy-3-nitrobenzene (14)

To a mixture of 13 (54.8 mg, 0.22 mmol), TBAI (81.3 mg, 0.22 mmol), KOH (24.7 mg, 0.44 mmol) in DMF (2.2 ml) was added Mel (0.014 ml, 0.22 mmol) and stirred at 40° C. for 48 h. ice water was added and the precipitant was collected, washed with water, and dried under vacuum. The product 14 (81%) was afforded as a yellow solid. ¹H-NMR (600 MHz, CDCl₃) δ 7.47 (s, 1H), 7.21 (s, 1H), 3.96 (s, 3H), 3.93 (s, 3H). ¹³C-NMR (150 MHz, CDCl₃) δ 154.88, 145.28, 142.29, 119.40, 118.80, 115.87, 62.16, 56.81.

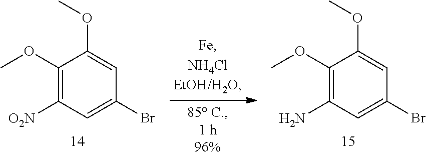

### 5-bromo-2,3-dimethoxyaniline (15)

To a mixture of 14 (184.4 mg, 0.71 mmol) and NH₄Cl (199.5 mg, 3.76 mmol) in EtOH/H₂O (5:1, 5 ml) was added iron powder (198.2 mg, 3.55 mmol) and stirred at 85° C. for 1 h. The reaction mixture was allowed to cool to room temperature, filtered through celite, and the filtrate was concentrated. The crude product was separated by chromatography (5-10% EtOAc in hexane) and yielded 15 (96%) as a yellow oil. ¹H-NMR (600 MHz, CDCl₃) δ 6.53 (d, J=2.1 Hz, OH), 6.45 (d, J=1.7 Hz), 3.90 (s, 1H), 3.82 (s, 1H), 3.79 (s, 1H). ¹³C-NMR (150 MHz, CDCl₃) δ 153.38, 141.61, 134.75, 116.69, 111.49, 105.73, 59.85, 55.90.

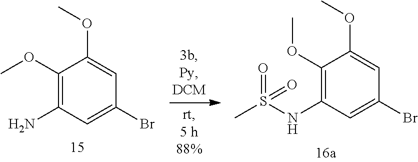 [SMILES: COc1cc(Br)cc(N)c1OC ; COc1cc(Br)cc(NS(C)(=O)=O)c1OC]

### N-(5-bromo-2,3-dimethoxyphenyl)methanesulfonamide (16a)

Method B; The crude product was separated by chromatography (15-20% EtOAc in hexane), and yielded 16a (88%) as a white solid. ¹H-NMR (600 MHz, CDCl₃) δ 7.34 (s, 1H), 7.03 (s, 1H), 6.84 (s, 1H), 3.87 (s, 6H), 3.04 (s, 3H). ¹³C-NMR (150 MHz, CDCl₃) δ 152.88, 136.96, 131.83, 117.04, 113.69, 111.78, 60.99, 56.15, 39.64.

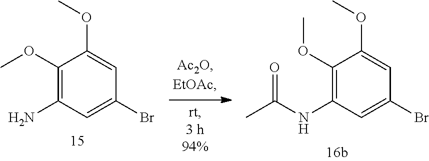

### N-(5-bromo-2,3-dimethoxyphenyl)methanesulfonamide (16b)

To a solution of 15 (15 mg, 0.065 mml) in EtOAc (0.5 ml) was added Ac₂O (0.018 ml, 0.19 mmol) and stirred at room temperature for 3 h. The reaction mixture was quenched with saturated aqueous Na₂HCO₃ and extracted with EtOAc. The combined organic phases were washed with brine, dried over anhydrous Na₂SO₄, and concentrated. The crude product was separated by chromatography (15-30% EtOAc in hexane), and yielded 16b (94%) as white solid. ¹H-NMR (600 MHz, CDCl₃) δ 8.23 (d, J=1.7 Hz, 1H), 7.78 (s, 1H), 6.80 (d, J=2.1 Hz, 1H), 3.86 (d, J=1.5 Hz, 7H), 2.21 (s, 3H). ¹³C-NMR (150 MHz, CDCl₃) δ 168.3, 152.2, 136.1, 132.8, 116.8, 115.2, 110.9, 60.7, 56.0, 24.9.

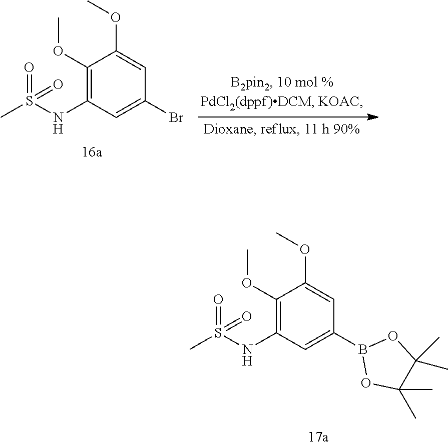

### N-(2,3-dimethoxy-5-(4,4,5,5-tetramethyl-1,3,2-dioxaborolan-2-yl)phenyl)methanesulfonamide (17a)

A mixture of 16a (23.6 mg, 0.076 mmol), B2Pin2 (23.2 mg, 0.092 mmol), PdCl₂(dppf)·DCM (9.3 mg, 0.012), and KOAc (22.7 mg, 0.23 mmol) in anhydrous Dioxane (0.7 ml) was stirred at 100° C. for 11 h under argon. The reaction was quenched with water and the aqueous layer was extracted twice with EtOAc. The combined organic phases were washed with brine, dried over anhydrous Na₂SO₄, and concentrated. The crude product was separated by chromatography (10-20% EtOAc in hexane), and yielded 17a (90%) as a white solid. ¹H-NMR (600 MHz, CDCl₃) δ 7.57 (s, 1H), 7.14 (s, 1H), 6.94 (s, 1H), 3.92 (d, J=4.1 Hz, 6H), 3.04 (s, 3H), 1.33 (s, 12H). ¹³C-NMR (150 MHz, CDCl₃) δ 151.83, 140.85, 130.48, 117.96, 114.40, 84.13, 61.05, 56.06, 39.69, 24.93.

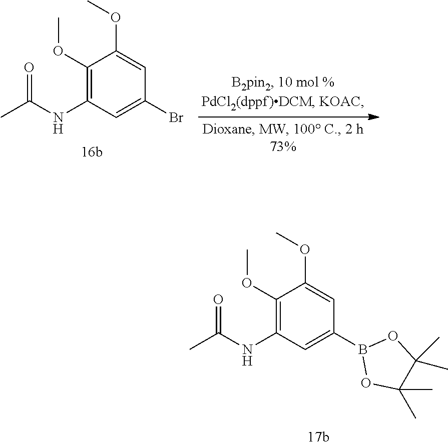 [SMILES: COc1cc(Br)cc(NC(C)=O)c1OC ; COc1cc(B2OC(C)(C)C(C)(C)O2)cc(NC(C)=O)c1OC]

### N-(2,3-dimethoxy-5-(4,4,5,5-tetramethyl-1,3,2-dioxaborolan-2-yl)phenyl)acetamide (17b)

A mixture of 16b (10 mg, 0.027 mmol), B₂Pin₂ (10.3 mg, 0.04 mmol), PdCl₂(dppf)·DCM (13.21 mg, 0.016), and KOAc (7.94 mg, 0.081 mmol) in anhydrous Dioxane (0.5 ml) was stirred at 100° C. in MW under argon. The reaction was quenched with water and aqueous layer was extracted twice with EtOAc. The combined organic phases were washed with brine, dried over anhydrous Na₂SO₄, and concentrated. The crude product was separated by chromatography (10-20% EtOAc in hexane), and yielded 17b (73%) as yellow oil. ¹H-NMR (600 MHz, CDCl₃) δ 8.35 (s, 1H), 7.76 (s, 1H), 7.09 (s, 1H), 3.90 (d, J=10.8 Hz, 6H), 1.32 (s, 12H). ¹³C-NMR (150 MHz, CDCl₃) δ 168.14, 151.26, 139.94, 131.50, 119.39, 113.26, 83.81, 74.98, 60.68, 55.85, 24.84, 24.80.

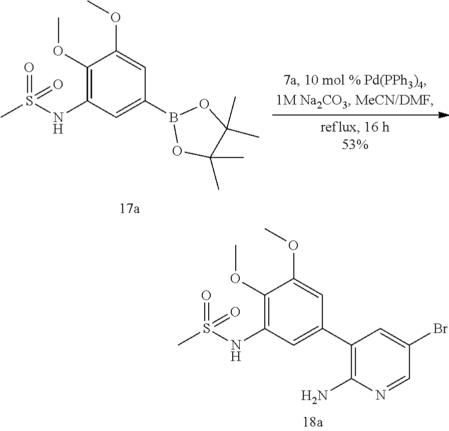

### N-(5-(2-amino-5-bromopyridin-3-yl)-2,3-dimethoxyphenyl)methanesulfonamide (18a)

Method D; The crude product was separated by chromatography (40% EtOAc in hexane), and yielded 18a (53%) as a light yellow solid. ¹H-NMR (600 MHz, CDCl₃) δ 8.11 (d, J=1.4 Hz, 1H), 7.47 (d, J=1.4 Hz, 1H), 7.22 (s, 1H), 7.05 (s, 1H), 6.75 (s, 1H), 4.65 (s, 2H), 3.95 (s, 3H), 3.91 (s, 3H), 3.06 (s, 3H), 1.58 (s, 2H). ¹³C-NMR (150 MHz, CDCl₃) δ 154.44, 152.83, 148.01, 139.60, 137.81, 133.00, 131.41, 122.50, 111.13, 108.61, 108.37, 61.07, 56.10, 40.04.

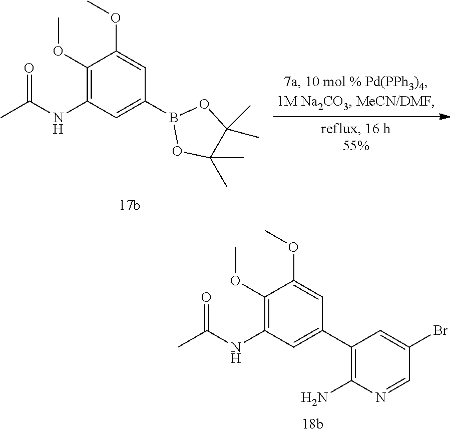 [SMILES: COc1cc(B2OC(C)(C)C(C)(C)O2)cc(NC(C)=O)c1OC ; COc1cc(-c2cc(Br)cnc2N)cc(NC(C)=O)c1OC]

### N-(5-(2-amino-5-bromopyridin-3-yl)-2,3-dimethoxyphenyl)acetamide (18b)

Method D; The crude product was separated by chromatography (60% EtOAc in hexane), and yielded 18b (55%) as light yellow oil. ¹H-NMR (600 MHz, CDCl₃) δ 8.08 (d, J=1.9 Hz, 2H), 7.87 (s, 1H), 7.48 (d, J=2.2 Hz, 1H), 6.70 (d, J=1.9 Hz, 1H), 4.72 (s, 2H), 3.93 (s, 3H), 3.90 (s, 3H), 2.23 (s, 3H). ¹³C-NMR (150 MHz, CDCl₃) δ 168.49, 154.60, 152.20, 147.53, 139.60, 132.55, 132.49, 123.19, 112.50, 108.15, 107.71, 60.83, 55.96, 24.92.

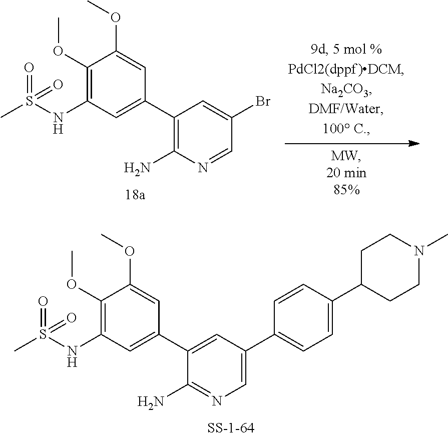

### N-(5-(2-amino-5-(4-(1-methylpiperidin-4-yl)phenyl)pyridin-3-yl)-2,3-dimethoxyphenyl)methanesulfonamide (SS-1-64)

### (Method H)

To a mixture of 18a (20 mg, 0.05 mmol), 9d (14.97 mg, 0.05 mmol), Pd(dppf)Cl₂·DCM (2.04 mg, 0.0025 mmol), and Na₂CO₃ (21.2, 0.2 mmol) in anhydrous DMF (1 ml) and DI-water (0.27 ml) under argon at 100° C. for 20 min under microwave irradiation. The reaction was quenched with water and the aqueous layer was extracted with DCM multiple times. The combined organic phases were washed with brine, dried over anhydrous Na₂SO₄, and concentrated. The crude product was separated by chromatography (5% MeOH in DCM), and yielded SS-1-64 (85%) as a yellow solid. mp 202-204° C. ¹H-NMR (600 MHz, CDCl₃) δ 8.31 (d, J=1.4 Hz, 1H), 7.58 (d, J=1.4 Hz, 1H), 7.48 (d, J=7.9 Hz, 2H), 7.28-7.30 (m, 3H), 6.83 (s, 1H), 4.74 (s, 2H), 3.95 (s, 3H), 3.91 (s, 3H), 3.07 (s, 3H), 3.01 (d, J=11.4 Hz, 2H), 2.50-2.55 (m, 1H), 2.34 (s, 3H), 2.07-2.11 (m, 2H), 1.82-1.88 (m, 4H). ¹³C-NMR (150 MHz, CDCl₃) δ 154.78, 152.77, 145.59, 145.18, 137.70, 136.31, 135.89, 134.27, 131.33, 127.73, 127.43, 126.38, 120.80, 111.60, 108.84, 61.02, 56.27, 56.02, 46.39, 41.60, 40.01, 33.39. HRMS (ESI) m/z: [M+H]⁺ calculated for C₂₆H₃₂N₄O₄S: 497.2217; observed 497.2222. Purity 99%, t_{R}=16.4 min.

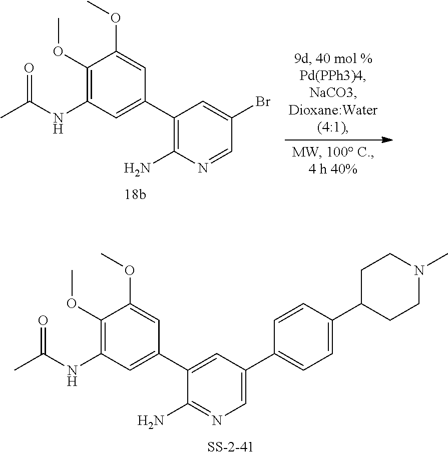 [SMILES: COc1cc(-c2cc(Br)cnc2N)cc(NC(C)=O)c1OC ; COc1cc(-c2cc(-c3ccc(C4CCN(C)CC4)cc3)cnc2N)cc(NC(C)=O)c1OC]

### N-(5-(2-amino-5-(4-(1-methylpiperidin-4-yl)phenyl)pyridin-3-yl)-2,3-dimethoxyphenyl)acetamide (SS-2-41)

Method H. The crude product was separated by chromatography (5% MeOH in DCM), and yielded SS-2-41 (40%) as a white solid. mp 75-77° C. ¹H-NMR (600 MHz, CDCl₃) δ 8.28 (d, J=2.4 Hz, 1H), 8.13 (d, J=1.5 Hz, 1H), 7.85 (s, 1H), 7.59 (d, J=2.4 Hz, 1H), 7.46 (d, J=8.3 Hz, 2H), 7.26-7.28 (m, 2H), 6.76 (d, J=1.7 Hz, 1H), 4.71 (s, 2H), 3.92 (s, 3H), 3.88 (s, 3H), 2.98 (d, J=11.5 Hz, 2H), 2.48-2.51 (m, 1H), 2.32 (s, 3H), 2.22 (s, 3H), 2.05 (td, J=11.4, 3.4 Hz, 2H), 1.80-1.86 (m, 4H). ¹³C-NMR (150 MHz, CDCl₃) δ 168.42, 154.90, 152.15, 145.29, 136.62, 136.38, 136.08, 133.94, 132.45, 127.61, 127.41, 126.39, 121.47, 112.65, 107.92, 60.86, 56.30, 55.96, 46.42, 41.62, 33.40, 25.00. HRMS (ESI) m/z: [M+H]⁺ calculated for C₂₆H₃₂N₄O₄S: 461.2547; observed 461.2552. Purity 99%, t_{R}=16.53 min.

### Example 2: Receptor Interacting Protein Kinase 2 (RTPK2) Enzyme Assay

Recombinant RIPK2 protein (20 ng per reaction) is diluted in the reaction buffer consisting of 40 mM Tris (pH 7.5); 20 mM MgCl₂; 0.1 mg/ml BSA; 50 μM DTT. Diluted protein is added to low volume white 384 well plates (2 μL/well). Inhibitors are diluted in reaction buffer (final 25% DMSO), 1 μL is added to each well and incubated 5 min at room temperature. Reactions are initiated by the addition of 2 μL of 100 μM ATP and 1 mg/ml RS repeat peptide (SignalChem) in the reaction buffer. Plates are sealed with plastic coverslips and incubated at room temperature for 2 h. Reactions are stopped by the addition of 5 μL of ADP-Glo reagent (Promega) and ADP generation reaction is performed for 40 min at room temperature. Luminescence signal is generated by the addition of 10 μL of Kinase detection reagent (Promega) for 30 min at room temperature. Luminescence signals are determined using appropriate luminescence plate-reader (typical integration time 0.3-1 sec). To calculate percent inhibition, average background signal is subtracted from test well and maximal signal wells. Inhibition, %=(1−(test signal/maximal signal))*100. The percent inhibition at a specified concentration is determined or IC₅₀ values are calculated based on a dose range of inhibitor concentrations using non-linear regression in GraphPad Prism software.

### Example 3: NOD2 Signaling Assay #1

HEK-Blue cells expressing human NOD2 and NFkB-SAEP reporter (Invivogen) are seeded into 96 well clear plates at 7.5×10³ cells per well in 100 μL of DMEM media supplemented with 10% FBS and 1% antibiotic-antimycotic mix. Cells are allowed to attach for 48 h in 5% CO₂ tissue culture incubator at 37° C. On the morning of the experiment, media in the wells is replaced with 100 μL of HEK-Blue detection media (Invivogen). Cells are treated with the inhibitors, diluted in DMSO (0.5 μL per well) for 15 min in 5% CO₂ tissue culture incubator at 37° C. After that, cells are stimulated by the addition of 1 ng/ml L18-MDP (Invivogen). Cells are incubated in 5% CO₂ tissue culture incubator at 37° C. for 8 h and absorbance, corresponding to the SEAP in the media, is determined in Wallac3V plate reader (Perkin Elmer). Inhibition, %=(1−((sample signal-unstimulated and DMSO treated cells)/(L18-MDP stimulated and DMSO treated cells−unstimulated and DMSO treated cells)))*100. IC₅₀ values are calculated based on a dose range of inhibitor concentrations using non-linear regression in GraphPad Prism software.

### Example 4: NOD2 Signaling Assay #2

THP1 cells expressing NFkB-SAEP reporter (Invivogen) are seeded into 96 well clear plates at 5×10⁴ cells per well in 100 μL of RPM11640 media supplemented with 10% FBS and 1% antibiotic-antimycotic mix. Cells are treated with the inhibitors, diluted in DMSO (0.5 mL per well) for 15 min in 5% CO₂ tissue culture incubator at 37° C. After that, cells are stimulated by the addition of 10 ng/ml L18-MDP (Invivogen). Cells are incubated in 5% CO₂ tissue culture incubator at 37° C. for 8 h and absorbance, corresponding to the SEAP in the media, is determined in Wallac3V plate reader (Perkin Elmer). Inhibition, %=(1−((sample signal-unstimulated and DMSO treated cells)/(L18-MDP stimulated and DMSO treated cells−unstimulated and DMSO treated cells)))*100. IC₅₀ values are calculated based on a dose range of inhibitor concentrations using non-linear regression in GraphPad Prism software.

### Example 5: Inhibition of RIPK2 and NOD2 Cellular Signaling by Compounds

Prepared compounds were evaluated for their ability to inhibit RIPK2 and NOD2 cellular signaling using the methods described above. The percent inhibition at a specified concentration or IC₅₀ values for inhibition of RIPK2 enzyme and NOD2 cellular signaling by the compounds are shown in Table 1.

TABLE 1
Inhibition data of RIPK2 and NOD2
cellular signaling by compounds.

| | IC₅₀ (nM) | | |
|---|---|---|---|
| Compound | Enzyme | HEKBlue | THP1 |
| SS-1-5 | NI | 57 | NT |
| SS-1-10 | NI | 16 | NT |
| SS-1-14 | 11 | 0.06 | NT |
| SS-1-18 | 10 | 0.03 | NT |
| SS-1-33 | 29 | 0.17 | NT |
| SS-1-38 | 31 | 0.07 | NT |
| SS-1-39 | 137 | 1.2 | NT |
| SS-1-43 | NT | NT | 0.1 |
| SS-1-50 | NT | NT | 1.2 |
| SS-1-64 | NT | NT | 0.014 |
| SS-1-66 | NT | NT | 20 |
| SS-2-41 | NT | NT | 0.10 |

NI: no inhibition observed; NT: not tested.

### Example 6: Pharmacokinetic Analysis of SS-1-64

After a single dose of 10 mg/kg of SS-1-64 administrated intraperitoneally [6% Captisol® in water formulation] to 8-week-old female C57BL/6 mice (n=5 mice/per group), the maximum plasma concentration (Cmax) of 1.63±1.2 μM was reached in 1 h (Tmax) with an AUC of 173.95±18.12 min·mg/mL, a plasma elimination half-life (t_{1/2}) of 7.88±0.28 h, clearance (CL) of 53.56±5.68 mL/min/kg and a volume of distribution of 36.45±2.61 L/kg. The pharmacokinetic properties of SS-1-64 are summarized in the following table along with a comparator RIPK2/NOD inhibitor CSLP37, N-(5-(2-Amino-5-(4-(piperazin-1-yl)phenyl)pyridin-3-yl)-3-fluoro-2-methoxyphenyl)propane-1-sulfonamide (U.S. Pat. No. 11,135,298 to Cuny et al.).

TABLE 2
Pharmacokinetic Analysis of SS-1-64

| | CSLP37 | SS-1-64 |
|---|---|---|
| Cₘₐₓ (ng/mL) | 0.55 ± 0.2 | 1.63 ± 1.2 |
| Tₘₐₓ (min) | 72 ± 26.83 | 60 ± 0 |
| AUC (min · mg/mL) | 175.22 ± 59.94 | 173.95 ± 18.12 |
| Cl (mL/min/kg) | 59.66 ± 15.09 | 53.56 ± 5.68 |
| V (L/kg) | 24.04 ± 6.15 | 36.45 ± 2.61 |
| t_{1/2} (h) | 4.66 ± 0.24 | 7.88 ± 0.28 |

### Example 7: Analysis of SS-1-64 in Experimental Autoimmune Encephalomyelitis (EAE) Model of Multiple Sclerosis

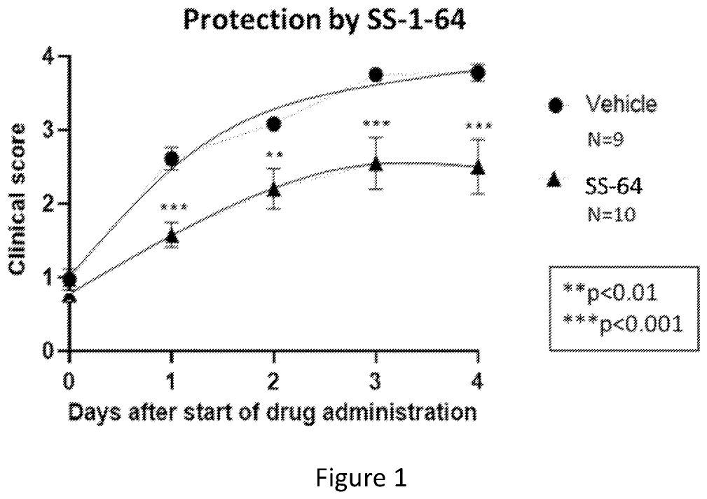

Female C57BL/6J mice, at 8-10 weeks of age, were immunized with an emulsion of MOG₃₅₋₅₅ peptide (10 mg/mL; Syd Labs) in 5 mg/mL complete Freund's adjuvant (CFA; Thermo Fisher #77140). The emulsion was delivered by two subcutaneous injections to the thighs in a volume of 100 μL per injection site. Bordetella pertussis toxin (PTX; Hooke Labs BT-0105) was administered via intraperitoneal injection on Day 0 and Day 1 at a dose of 260 ng/animal in 100 L of PBS. Following EAE induction, mice were monitored daily for paralytic symptoms and scored for their clinical presentation using a progressive scoring system (Score 0: no disease; Score 1: flaccid tail; Score 2: hindlimb weakness; Score 3: partial hindlimb paralysis; Score 4: complete bilateral hindlimb paralysis; Score 5: death). Animals were dosed starting at clinical score of 0.5-1. Drug dosing (25 mg/kg per injection) was done orally in 20% Captisol/saline twice daily. Results are shown in the following graph of SS-1-64 versus vehicle control, where the x-axis displays the number of days following the initiation SS-1-64 administration until the animals were sacrificed. The y-axis represents the severity of symptoms each day (FIG. 1).

1. A compound of formula 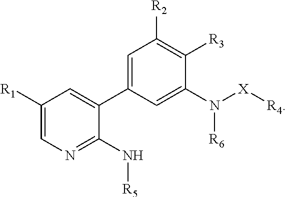
or a pharmaceutically acceptable salt thereof,
wherein—
R₁ is selected from 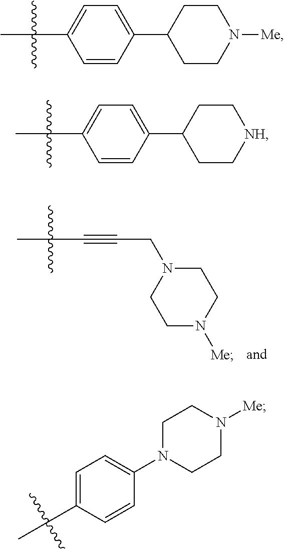
R₂ is selected from H, halo, C₁-C₃ alkoxyl optionally substituted with halo, and —O-phenyl;
R₃ is selected from H, halo, C₁-C₃ alkoxyl optionally substituted with halo, and C₁-C₃ alkyl;
R₄ is selected from C₁-C₃ alkyl optionally substituted with halo, phenyl, and benzyl;
R₅ is selected from H and methyl;
R₆ is selected from H and methyl; and
X is selected from SO₂ and CO or
wherein—
R₁ is selected from 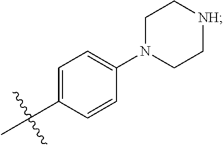 [SMILES: *c1ccc(N2CCNCC2)cc1]
R₂ is selected from H, halo, C₁-C₃ alkoxyl optionally substituted with halo, and —O-phenyl;
R₃ is selected from H, halo, C₁-C₃ alkoxyl optionally substituted with halo, and C₁-C₃ alkyl;
R₄ is selected from C₁-C₃ alkyl optionally substituted with halo, phenyl, and benzyl;
R₅ is selected from H and methyl;
R₆ is selected from H and methyl;
X is selected from SO₂ and CO and
(i) at least one of R₅ or R₆ is methyl or (ii) if each of R₅ and R₆ are hydrogen, then R₄ is methyl or X is CO.

2. The compound of claim 1, wherein R₁ is

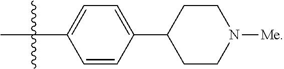 [SMILES: *c1ccc(C2CCN(C)CC2)cc1]

3. The compound of claim 1, wherein R₁ is

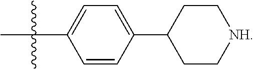 [SMILES: *c1ccc(C2CCNCC2)cc1]

4. The compound of claim 1, wherein R₁ is

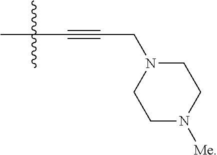 [SMILES: *C#CCN1CCN(C)CC1]

5. The compound of claim 1, wherein R₁ is

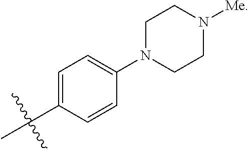 [SMILES: *c1ccc(N2CCN(C)CC2)cc1]

6. The compound of claim 1, wherein R₁ is

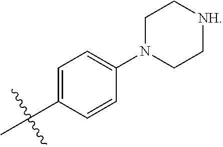 [SMILES: *c1ccc(N2CCNCC2)cc1]

7. The compound of any one of claims 1-6, wherein R₂ is methoxy.

8. The compound of any one of claims 1-6, wherein R₃ is methoxy or fluoro.

9. The compound of any one of claims 1-6, wherein each of R₂ and R₃ are methoxy.

10. The compound of any one of claims 1-6, wherein R₂ is fluoro and R₃ is methoxy.

11. The compound of any one of claims 1-10, wherein R₄ is methyl or propyl.

12. The compound of any one of claims 1-11, wherein R₅ is methyl or hydrogen.

13. The compound of any one of claims 1-12, wherein R₆ is methyl or hydrogen.

14. The compound of any one of claims 1-13, wherein X is CO.

15. The compound of any one of claims 1-13, wherein X is SO₂.

16. The compound of claim 1, wherein the compound is selected from:
N-(3-fluoro-2-methoxy-5-(2-(methylamino)-5-(4-(piperazin-1-yl)phenyl)pyridin-3-yl)phenyl)propane-1-sulfonamide,
N-(5-(2-amino-5-(4-(piperidin-4-yl)phenyl)pyridin-3-yl)-3-fluoro-2-methoxyphenyl)propane-1-sulfonamide,
N-(5-(2-amino-5-(4-(4-methylpiperazin-1-yl)phenyl)pyridin-3-yl)-3-fluoro-2-methoxyphenyl)propane-1-sulfonamide,
N-(5-(2-amino-5-(4-(piperazin-1-yl)phenyl)pyridin-3-yl)-3-fluoro-2-methoxyphenyl)methanesulfonamide,
N-(5-(2-amino-5-(4-(1-methylpiperidin-4-yl)phenyl)pyridin-3-yl)-3-fluoro-2-methoxyphenyl)propane-1-sulfonamide,
N-(5-(2-amino-5-(4-(piperazin-1-yl)phenyl)pyridin-3-yl)-3-fluoro-2-methoxyphenyl)-N-methylpropane-1-sulfonamide,
N-(5-(2-amino-5-(4-(1-methylpiperidin-4-yl)phenyl)pyridin-3-yl)-3-fluoro-2-methoxyphenyl)methanesulfonamide,
N-(5-(2-amino-5-(4-(1-methylpiperidin-4-yl)phenyl)pyridin-3-yl)-3-fluoro-2-methoxyphenyl)-N-methylmethanesulfonamide,
N-(5-(2-amino-5-(4-(1-methylpiperidin-4-yl)phenyl)pyridin-3-yl)-2,3-dimethoxyphenyl)methanesulfonamide,
N-(5-(2-amino-5-(3-(4-methylpiperazin-1-yl)prop-1-yn-1-yl)pyridin-3-yl)-3-fluoro-2-methoxyphenyl)propane-1-sulfonamide, and
N-(5-(2-amino-5-(4-(1-methylpiperidin-4-yl)phenyl)pyridin-3-yl)-2,3-dimethoxyphenyl)acetamide.

17. The compound of claim 1, wherein the compound is selected from:
N-(5-(2-amino-5-(4-(piperidin-4-yl)phenyl)pyridin-3-yl)-3-fluoro-2-methoxyphenyl)propane-1-sulfonamide,
N-(5-(2-amino-5-(4-(4-methylpiperazin-1-yl)phenyl)pyridin-3-yl)-3-fluoro-2-methoxyphenyl)propane-1-sulfonamide,
N-(5-(2-amino-5-(4-(piperazin-1-yl)phenyl)pyridin-3-yl)-3-fluoro-2-methoxyphenyl)methanesulfonamide,
N-(5-(2-amino-5-(4-(1-methylpiperidin-4-yl)phenyl)pyridin-3-yl)-3-fluoro-2-methoxyphenyl)propane-1-sulfonamide,
N-(5-(2-amino-5-(4-(1-methylpiperidin-4-yl)phenyl)pyridin-3-yl)-3-fluoro-2-methoxyphenyl)methanesulfonamide,
N-(5-(2-amino-5-(4-(1-methylpiperidin-4-yl)phenyl)pyridin-3-yl)-2,3-dimethoxyphenyl)methanesulfonamide, and
N-(5-(2-amino-5-(4-(1-methylpiperidin-4-yl)phenyl)pyridin-3-yl)-2,3-dimethoxyphenyl)acetamide.

18. The compound of claim 1, wherein the compound is selected from:
N-(5-(2-amino-5-(4-(piperidin-4-yl)phenyl)pyridin-3-yl)-3-fluoro-2-methoxyphenyl)propane-1-sulfonamide,
N-(5-(2-amino-5-(4-(4-methylpiperazin-1-yl)phenyl)pyridin-3-yl)-3-fluoro-2-methoxyphenyl)propane-1-sulfonamide,
N-(5-(2-amino-5-(4-(1-methylpiperidin-4-yl)phenyl)pyridin-3-yl)-3-fluoro-2-methoxyphenyl)propane-1-sulfonamide, and
N-(5-(2-amino-5-(4-(1-methylpiperidin-4-yl)phenyl)pyridin-3-yl)-2,3-dimethoxyphenyl)methanesulfonamide.

19. The compound of claim 1, wherein the compound is selected from:
N-(5-(2-amino-5-(4-(piperidin-4-yl)phenyl)pyridin-3-yl)-3-fluoro-2-methoxyphenyl)propane-1-sulfonamide,
N-(5-(2-amino-5-(4-(1-methylpiperidin-4-yl)phenyl)pyridin-3-yl)-3-fluoro-2-methoxyphenyl)propane-1-sulfonamide,
N-(5-(2-amino-5-(4-(1-methylpiperidin-4-yl)phenyl)pyridin-3-yl)-3-fluoro-2-methoxyphenyl)methanesulfonamide,
N-(5-(2-amino-5-(4-(1-methylpiperidin-4-yl)phenyl)pyridin-3-yl)-3-fluoro-2-methoxyphenyl)-N-methylmethanesulfonamide, and
N-(5-(2-amino-5-(4-(1-methylpiperidin-4-yl)phenyl)pyridin-3-yl)-2,3-dimethoxyphenyl)methanesulfonamide.

20. A pharmaceutical composition comprising the compound or pharmaceutically acceptable salt thereof according to any one of claims 1-19.

21. A method for treating an autoimmune or inflammatory disease in a subject, the method comprising administering the compound or pharmaceutically acceptable salt thereof according to claim 1 to the subject.

22. The method of claim 21, wherein the autoimmune or inflammatory disease is multiple sclerosis.

23. A method for inhibiting a protein kinase, the method comprising contacting the compound or pharmaceutically acceptable salt thereof according to claim 1 with the protein kinase.

24. The method of claim 23, wherein the protein kinase is receptor interacting protein kinase 2 (RIPK2).

25. The method of claim 24, wherein the compound or pharmaceutically acceptable salt is contacted with the RIPK2 in vivo.

26. The method of claim 25, wherein a subject is administered an effective amount of the compound or pharmaceutically acceptable salt to treat a disease or disorder associated with RIPK2 activity.

27. A method of inhibiting nucleotide-binding oligomerization domain (NOD) cell signaling, the method comprising contacting the compound or pharmaceutically acceptable salt thereof according to claim 1 with a cell expressing a NOD protein.

28. The method of claim 27, wherein the NOD protein is NOD2.

29. The method of claim 28, wherein the compound or pharmaceutically acceptable salt is contacted with the cell expressing NOD2 in vivo.

30. The method of claim 29, wherein a subject is administered an effective amount of the compound or pharmaceutically acceptable salt to treat a disease or disorder associated with NOD2 activity.

31. The method of any one of claims 21-30, wherein the compound is the compound according to any one of claims 2-15.

32. The method of any one of claims 21-30, wherein the compound is selected from:
N-(3-fluoro-2-methoxy-5-(2-(methylamino)-5-(4-(piperazin-1-yl)phenyl)pyridin-3-yl)phenyl)propane-1-sulfonamide,
N-(5-(2-amino-5-(4-(piperidin-4-yl)phenyl)pyridin-3-yl)-3-fluoro-2-methoxyphenyl)propane-1-sulfonamide,
N-(5-(2-amino-5-(4-(4-methylpiperazin-1-yl)phenyl)pyridin-3-yl)-3-fluoro-2-methoxyphenyl)propane-1-sulfonamide,
N-(5-(2-amino-5-(4-(piperazin-1-yl)phenyl)pyridin-3-yl)-3-fluoro-2-methoxyphenyl)methanesulfonamide,
N-(5-(2-amino-5-(4-(1-methylpiperidin-4-yl)phenyl)pyridin-3-yl)-3-fluoro-2-methoxyphenyl)propane-1-sulfonamide,
N-(5-(2-amino-5-(4-(piperazin-1-yl)phenyl)pyridin-3-yl)-3-fluoro-2-methoxyphenyl)-N-methylpropane-1-sulfonamide,
N-(5-(2-amino-5-(4-(1-methylpiperidin-4-yl)phenyl)pyridin-3-yl)-3-fluoro-2-methoxyphenyl)methanesulfonamide,
N-(5-(2-amino-5-(4-(1-methylpiperidin-4-yl)phenyl)pyridin-3-yl)-3-fluoro-2-methoxyphenyl)-N-methylmethanesulfonamide,
N-(5-(2-amino-5-(4-(1-methylpiperidin-4-yl)phenyl)pyridin-3-yl)-2,3-dimethoxyphenyl)methanesulfonamide,
N-(5-(2-amino-5-(3-(4-methylpiperazin-1-yl)prop-1-yn-1-yl)pyridin-3-yl)-3-fluoro-2-methoxyphenyl)propane-1-sulfonamide, and
N-(5-(2-amino-5-(4-(1-methylpiperidin-4-yl)phenyl)pyridin-3-yl)-2,3-dimethoxyphenyl)acetamide.

33. The method of any one of claims 21-30, wherein the compound is selected from:
N-(5-(2-amino-5-(4-(piperidin-4-yl)phenyl)pyridin-3-yl)-3-fluoro-2-methoxyphenyl)propane-1-sulfonamide,
N-(5-(2-amino-5-(4-(4-methylpiperazin-1-yl)phenyl)pyridin-3-yl)-3-fluoro-2-methoxyphenyl)propane-1-sulfonamide,
N-(5-(2-amino-5-(4-(piperazin-1-yl)phenyl)pyridin-3-yl)-3-fluoro-2-methoxyphenyl)methanesulfonamide,
N-(5-(2-amino-5-(4-(1-methylpiperidin-4-yl)phenyl)pyridin-3-yl)-3-fluoro-2-methoxyphenyl)propane-1-sulfonamide,
N-(5-(2-amino-5-(4-(1-methylpiperidin-4-yl)phenyl)pyridin-3-yl)-3-fluoro-2-methoxyphenyl)methanesulfonamide,
N-(5-(2-amino-5-(4-(1-methylpiperidin-4-yl)phenyl)pyridin-3-yl)-2,3-dimethoxyphenyl)methanesulfonamide, and
N-(5-(2-amino-5-(4-(1-methylpiperidin-4-yl)phenyl)pyridin-3-yl)-2,3-dimethoxyphenyl)acetamide.

34. The method of any one of claims 21-30, wherein the compound is selected from:
N-(5-(2-amino-5-(4-(piperidin-4-yl)phenyl)pyridin-3-yl)-3-fluoro-2-methoxyphenyl)propane-1-sulfonamide,
N-(5-(2-amino-5-(4-(4-methylpiperazin-1-yl)phenyl)pyridin-3-yl)-3-fluoro-2-methoxyphenyl)propane-1-sulfonamide,
N-(5-(2-amino-5-(4-(1-methylpiperidin-4-yl)phenyl)pyridin-3-yl)-3-fluoro-2-methoxyphenyl)propane-1-sulfonamide, and
N-(5-(2-amino-5-(4-(1-methylpiperidin-4-yl)phenyl)pyridin-3-yl)-2,3-dimethoxyphenyl)methanesulfonamide.

35. The method of any one of claims 21-30, wherein the compound is selected from:
N-(5-(2-amino-5-(4-(piperidin-4-yl)phenyl)pyridin-3-yl)-3-fluoro-2-methoxyphenyl)propane-1-sulfonamide,
N-(5-(2-amino-5-(4-(1-methylpiperidin-4-yl)phenyl)pyridin-3-yl)-3-fluoro-2-methoxyphenyl)propane-1-sulfonamide,
N-(5-(2-amino-5-(4-(1-methylpiperidin-4-yl)phenyl)pyridin-3-yl)-3-fluoro-2-methoxyphenyl)methanesulfonamide,
N-(5-(2-amino-5-(4-(1-methylpiperidin-4-yl)phenyl)pyridin-3-yl)-3-fluoro-2-methoxyphenyl)-N-methylmethanesulfonamide, and
N-(5-(2-amino-5-(4-(1-methylpiperidin-4-yl)phenyl)pyridin-3-yl)-2,3-dimethoxyphenyl)methanesulfonamide.
